(19) United States
(12) Patent Application Publication
(10) Pub. No.: US 2026/0258500 A1
(43) Pub. Date: Sep. 3, 2026

(54) BIOMARKER FOR METASTATIC BLADDER CANCER

(71) Applicant: DONG-A UNIVERSITY RESEARCH FOUNDATION FOR INDUSTRY-ACADEMY COOPERATION, Busan (KR)

(72) Inventors: Sun-Hee LEEM, Busan (KR); MI-So JEONG, Busan (KR); Jeong-Yeon MUN, Busan (KR); Gi-Eun YANG, Busan (KR)

(73) Assignee: DONG-A UNIVERSITY RESEARCH FOUNDATION FOR INDUSTRY-ACADEMY COOPERATION, Busan (KR)

(21) Appl. No.: 18/864,774

(22) PCT Filed: May 11, 2023
(86) PCT No.: PCT/KR2023/006419

§ 371 Date: Nov. 11, 2024

(30) Foreign Application Priority Data
May 11, 2022 (KR) 10-2022-0057725
May 11, 2023 (KR) 10-2023-0060957

(51) Int. Cl. C12Q 1/6886

(52) U.S. Cl. CPC C12Q 1/6886; C12Q 2600/118; C12Q 2600/136; C12Q 2600/158

(57) ABSTRACT

The present disclosure relates to a biomarker for metastatic bladder cancer. According to the present disclosure, it was confirmed that bladder cancer cell lines increase in infiltration and migration capacity while acquiring anti-cancer drug resistance, and it was confirmed that the expression of ATP8B1, CCND1, CDH12, FERMT1, HSPD1, HSPE1, ID3, MAML3, CDKNI A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA is increased in bladder cancer cells with increased infiltration and metastasis. In addition, it was confirmed that selected genes are associated with metastasis and prognosis of cancer in bladder cancer patients.

### CROSS-REFERENCE TO RELATED APPLICATIONS

This application is a national stage application of PCT/KR2023/006419 filed May 11, 2023 which claims priority to Korean Patent Application No. 10-2022-0057725 filed May 11, 2022 and Korean Patent Application No. 10-2023-0060957 filed May 11, 2023, the entire disclosures of which are incorporated herein by reference.

### INCORPORATION BY REFERENCE OF SEQUENCE LISTING

The content of the electronically submitted sequence listing, file name: Q303592_sequence listing as filed; size: 174,597 bytes; and date of creation: Nov. 11, 2024, filed herewith, is incorporated herein by reference in its entirety.

### TECHNICAL FIELD

The present disclosure relates to a biomarker for metastatic bladder cancer.

### BACKGROUND ART

Bladder cancer is a malignant tumor with the second highest mortality rate after prostate cancer among urological tumors. At least 90% of bladder cancer is transitional cell carcinoma, and approximately 60% thereof is superficial bladder cancer with low-grade differentiation. However, even after transurethral resection, recurrences are frequent, of which approximately 16 to 25% progresses to cancer with high-grade differentiation, and of which 10% develop stage progression and tumor metastasis. The frequent recurrence and the stage progression are frequently raised issues for bladder cancer patients and urologists, and therefore, there is a need to discover more effective prognostic indicators or develop treatments to prevent recurrence of bladder cancer and suppress the stage to invasiveness.

Despite much research to date, the exact cause of bladder cancer has not been identified, but smoking, exposure to carcinogens, and drug-taking have been reported as risk factors for bladder cancer. Important host factors for bladder cancer include mutations in primary oncogenes and tumor suppressor genes, heterozygous loss between specific alleles, methylation patterns of promoter sites of specific genes, and the like. The recurrence rate of non-muscle invasive bladder cancer (NMIBC) with a well-differentiated papillary Ta stage is 50 to 75%, and even if cancer recurrence is frequent, the progression to invasive bladder cancer that invades the muscle layer is less than 15%. On the other hand, most of a T1 stage invading the lamina propria is poorly differentiated, frequently accompanied by intraepithelial carcinoma, and progresses to invasive or metastatic cancer at 30 to 50% despite various treatments. In the poorly differentiated Ta stage, there is a 10 to 15% risk of stage progression compared to the well-differentiated Ta stage, but a risk lower than that of the T1 stage. In addition, at the time of diagnosis, approximately 30% is diagnosed as invasive tumors that have already invaded the muscle layer, and at least 50% thereof already has distant metastasis.

As such, bladder cancer shows various biodiversities, and clinical prognostic factors currently used include tumor differentiation, infiltration of the bladder lamina propria (tumor stage), vascular or lymphatic infiltration, the presence of intraepithelial carcinoma, etc., but even within the same pathological characteristics, biological diversity is not yet fully understood. It is very important in determining treatment protocol to predict the inherent biological characteristics and risk of recurrence or progression of superficial or invasive bladder cancer. In particular, it is more helpful in improving the patient's prognosis to select patients with high-risk of recurrence and progression among patients with superficial bladder cancer to perform more aggressive treatment.

Cytogenetic studies have found numerous structural changes and chromosomal abnormalities in bladder transitional cell carcinoma. Chromosomal abnormalities, such as an increase (hyperdiploidy) or decrease (aneuploidy) in the number of chromosomes, marker chromosomes, and changes in chromosome size or shape, and the like are closely related to an increased risk of recurrence and progression of bladder cancer. Molecular genetic studies of bladder cancer have found deletions of several chromosomal arms, particularly 3p, 6q, 9q, 11p, 17p, and 18q. The fact that 9q deletion is observed in both high- and low-differentiated bladder cancers suggests that 9q deletion may be the first alteration in the development of bladder cancer. In addition, deletions of 11p and 8p and additions of 8q and 1q are observed among early changes in superficial bladder cancer. Conversely, the fact that deletion of 17p is not observed in superficial bladder cancer, but approximately 60% of invasive bladder cancer shows loss of heterozygosity (LOH) of 17p suggests that the loss of the region is involved in the progression of bladder cancer.

Accordingly, the present inventors confirmed that cell invasion and migration increase according to changes in genes in a process of acquiring anticancer drug resistance of bladder cancer in non-muscle invasive bladder cancer cells, discovered key biomarkers whose expression changes in relation to invasion and metastasis of bladder cancer, and then completed the present disclosure.

### DISCLOSURE

### Technical Problem

An object of the present disclosure is to provide a biomarker of metastatic cancer including a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

Another object of the present disclosure is to provide a biomarker composition for predicting metastasis or prognosis of cancer, including a preparation for measuring the expression level of a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

Yet another object of the present disclosure is to provide a kit for predicting metastasis or prognosis of cancer including the composition.

Yet another object of the present disclosure is to provide a method of providing information for predicting metastasis or prognosis of cancer including: confirming the expression level of a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA; and comparing the expression level of the gene with a reference value of a control group.

Yet another object of the present disclosure is to provide a biomarker of cancer having drug resistance to gemcitabine including a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBB, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

Yet another object of the present disclosure is to provide a biomarker composition for diagnosis of cancer having drug resistance to gemcitabine, including a preparation for measuring the expression level of a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

### Technical Solution

In order to achieve the above aspects, the present disclosure provides a biomarker of metastatic cancer including a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

Further, the present disclosure provides a biomarker composition for predicting metastasis or prognosis of cancer, including a preparation for measuring the expression level of a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

Further, the present disclosure provides a kit for predicting metastasis or prognosis of cancer including the composition.

Further, the present disclosure provides a method of providing information for predicting metastasis or prognosis of cancer including: confirming the expression level of a gene selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA;
comparing the expression level of the gene with a reference value of a control group; and
determining that the cancer metastasis has increased when the expression level increases compared to the reference value of the control group.

Further, the present disclosure provides a biomarker of cancer having drug resistance to gemcitabine including a gene selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

Further, the present disclosure provides a biomarker composition for diagnosis of cancer having drug resistance to gemcitabine, including a preparation for measuring the expression level of a gene selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

### Advantageous Effects

According to the present disclosure, it was confirmed that bladder cancer cell lines increase in infiltration (invasion) and migration capacity while acquiring stage-specific anti-cancer drug resistance, and it was confirmed that the expression of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA is increased in bladder cancer cells with increased infiltration (invasion) and metastasis. In addition, selected genes are confirmed to be associated with metastasis and prognosis of cancer in bladder cancer patients, and thus can be usefully utilized in related industries.

### DESCRIPTION OF DRAWINGS

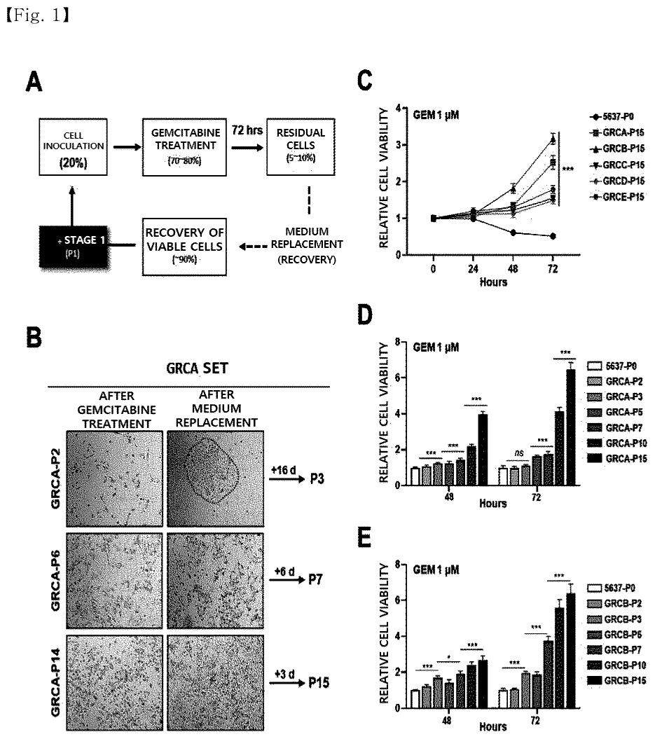

A-E of FIG. 1 are diagrams showing construction of a staged molecular evolution model of an anticancer drug-resistant bladder cancer cell line of the present disclosure and cell line viability after gemcitabine treatment.

A of FIG. 1: Schematic diagram of 5637GRC cell line construction process

B of FIG. 1: Images of stage-specific construction process of GRCA P3, P7, and P15 cell lines

C of FIG. 1: Confirmation of P15 cell viability of GRCA to GRCE according to gemcitabine treatment

D of FIG. 1: Confirmation of stage-specific cell viability to gemcitabine in GRCA cell lines

E of FIG. 1: Confirmation of stage-specific cell viability to gemcitabine in GRCB cell lines

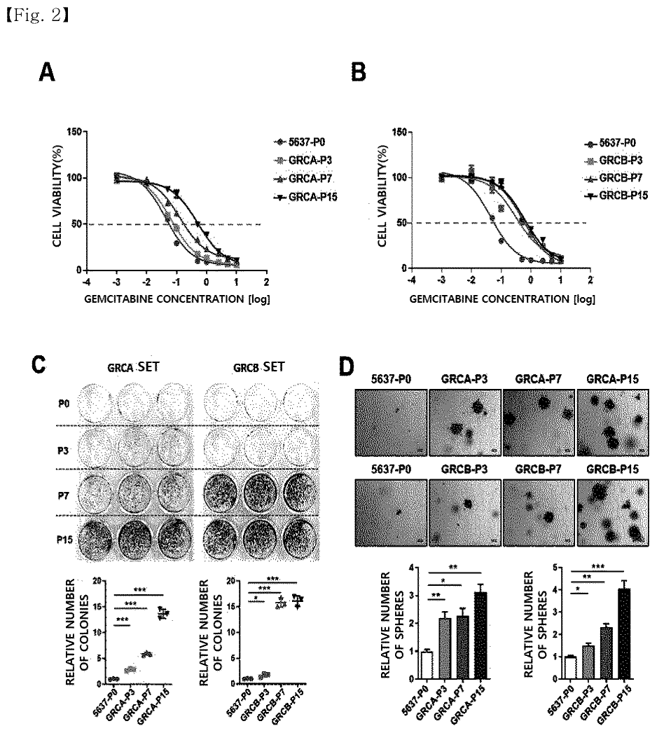

A-D of FIG. 2 are diagrams confirming that gemcitabine resistance increases stepwise in GRC cell lines of the present disclosure.

A of FIG. 2: Confirmation of IC₅₀ values for gemcitabine in GRCA cell lines

B of FIG. 2: Confirmation of IC₅₀ values for gemcitabine in GRCB cell lines

C of FIG. 2: Confirmation of colony formation capacity after gemcitabine treatment in GRCA and GRCB cell lines

D of FIG. 2: Confirmation of sphere formation after gemcitabine treatment in GRCA and GRCB cell lines

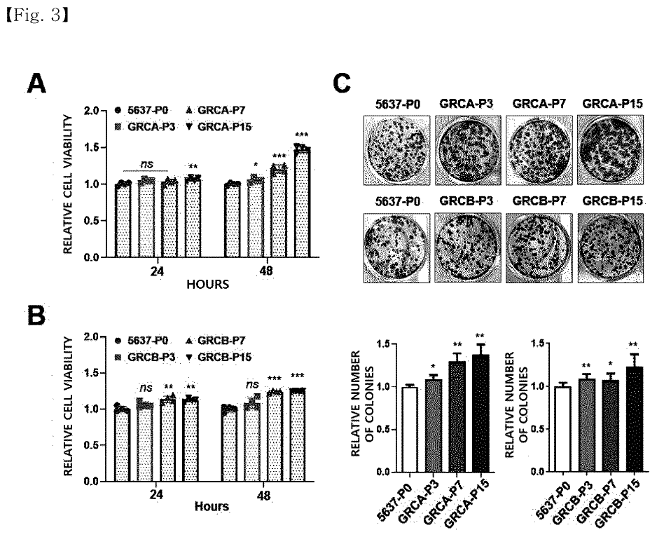

A-C of FIG. 3 are diagrams confirming stage-specific cell proliferation of GRC cell lines that are not treated with gemcitabine in the present disclosure.

A of FIG. 3: Confirmation of stage-specific cell proliferation in GRCA cell lines

B of FIG. 3: Confirmation of stage-specific cell proliferation in GRCB cell lines

C of FIG. 3: Confirmation of stage-specific colony formation in GRCA and GRCB cell lines

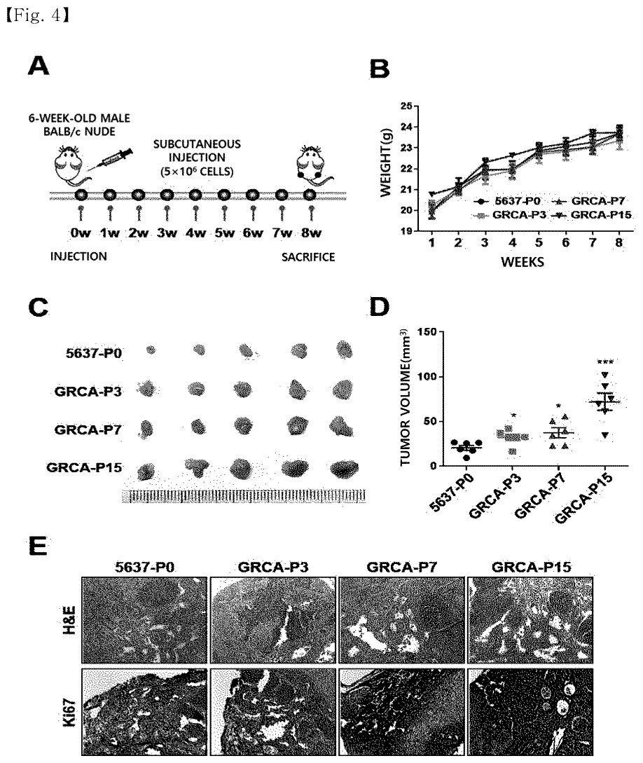

A-E of FIG. 4 are diagrams confirming tumor formation capacity of GRCA cell lines of the present disclosure in mice.

A of FIG. 4: Schematic diagram of tumor formation capacity experiment using mice

B of FIG. 4: Confirmation of weight changes in mice

C of FIG. 4: Visual observation of stage-specific tumor formation in GRCA cell lines

D of FIG. 4: Quantification of stage-specific tumor volumes in GRCA cell lines

E of FIG. 4: Stage-specific H&E and Ki67 staining results of tumors formed from GRCA cell lines

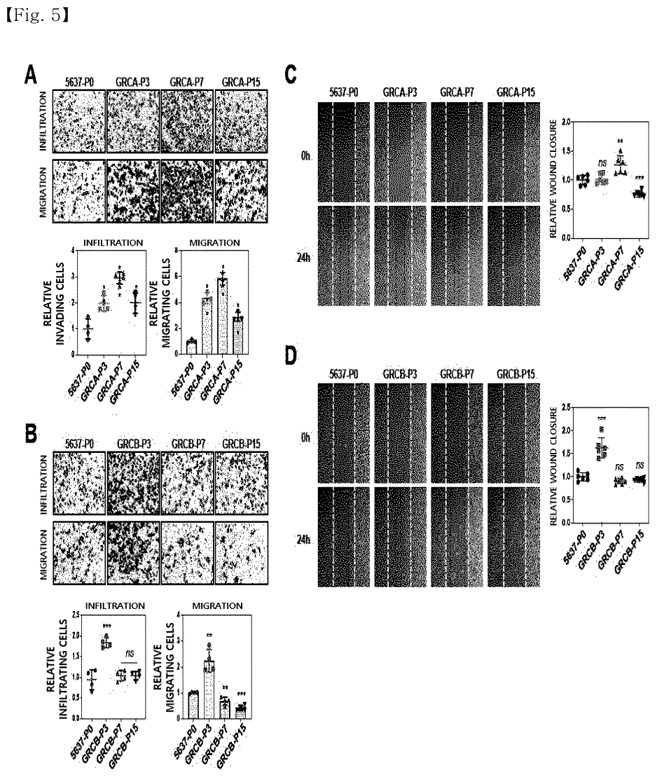

A-D of FIG. 5 are diagrams confirming cell motility according to acquisition of gemcitabine resistance in GRC cell lines of the present disclosure.

A of FIG. 5: Confirmation of infiltration (invasion) and migration capacity of GRCA cell lines using Boyden chamber assays

B of FIG. 5: Confirmation of infiltration (invasion) and migration capacity of GRCB cell lines using Boyden chamber assays

C of FIG. 5: Confirmation of stage-specific migration capacity of GRCA cell lines by wound healing assays

D of FIG. 5: Confirmation of stage-specific migration capacity of GRCB cell lines by wound healing assays

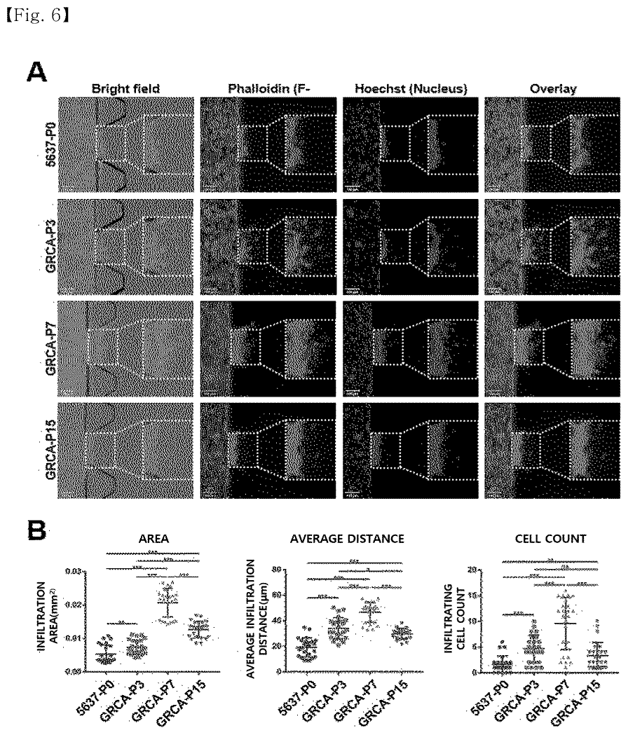

A-B of FIG. 6 are diagrams confirming cell migration capacity in 3D culture of GRCA cell lines of the present disclosure.

A of FIG. 6: Images of 3D culture of GRCA cell lines using microfluidic device

B of FIG. 6: Quantification of cell infiltration area, infiltration distance, and number of infiltrating cells

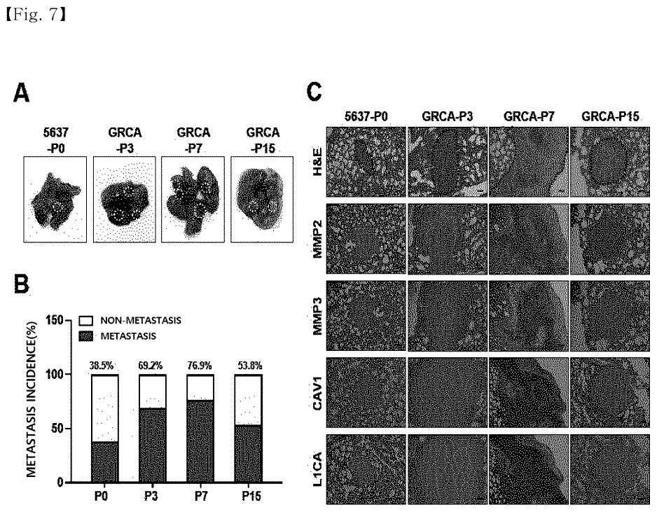

A-C of FIG. 7 are diagrams confirming metastatic capacity of GRCA cell lines of the present disclosure in mice.

A of FIG. 7: Confirmation of stage-specific metastatic capacity in GRCA cell lines

B of FIG. 7: Quantification of stage-specific metastasis incidence in GRCA cell lines

C of FIG. 7: Histochemical staining results of H&E, MMP2, MMP3, CAV1, and L1CAM in lung tissues metastasized by GRCA cell lines

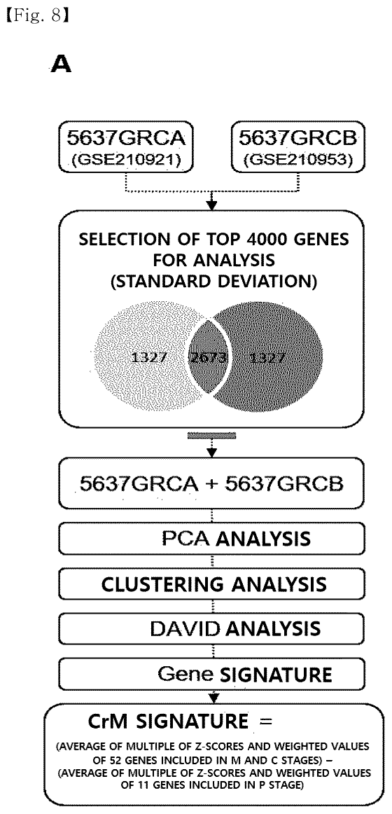

A of FIG. 8 is a diagram summarizing an analysis process of a molecular mechanism of GRC cell lines of the present disclosure.

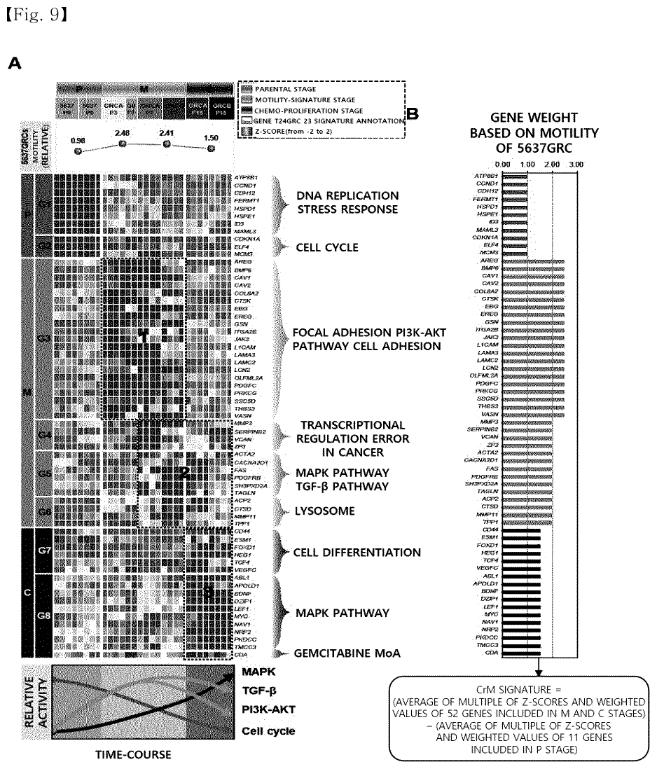

A-B of FIG. 9 are diagrams showing biological characteristics related to differentially expressed genes representing three stages of a GRC cell line of the present disclosure.

A of FIG. 9: Heatmap of 63 genes selected from differentially expressed genes commonly in GRCA and GRCB cell lines and relative activity in four biological pathways according to a time course

B of FIG. 9: CrM signature and weighted value calculation of 63 genes

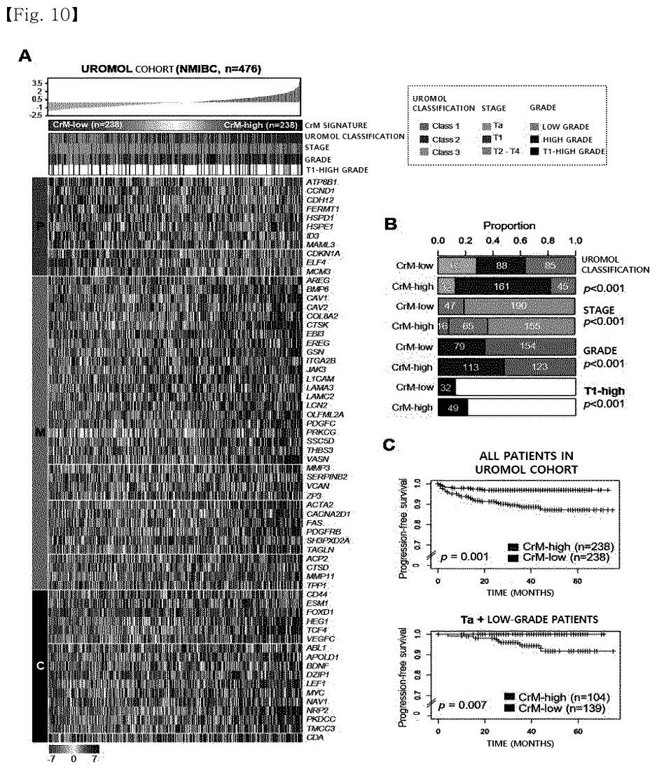

A-C of FIG. 10 are diagrams of gene expression patterns and survival analysis for NMIBC patients in a UROMOL cohort based on the CrM signature of the present disclosure.

A of FIG. 10: Heatmap of UROMOL cohort divided into two groups based on CrM signature

B of FIG. 10: Comparison of distribution and clinical information of UROMOL stratified by CrM signature

C of FIG. 10: Kaplan-Meier plot of two groups stratified by CrM signature

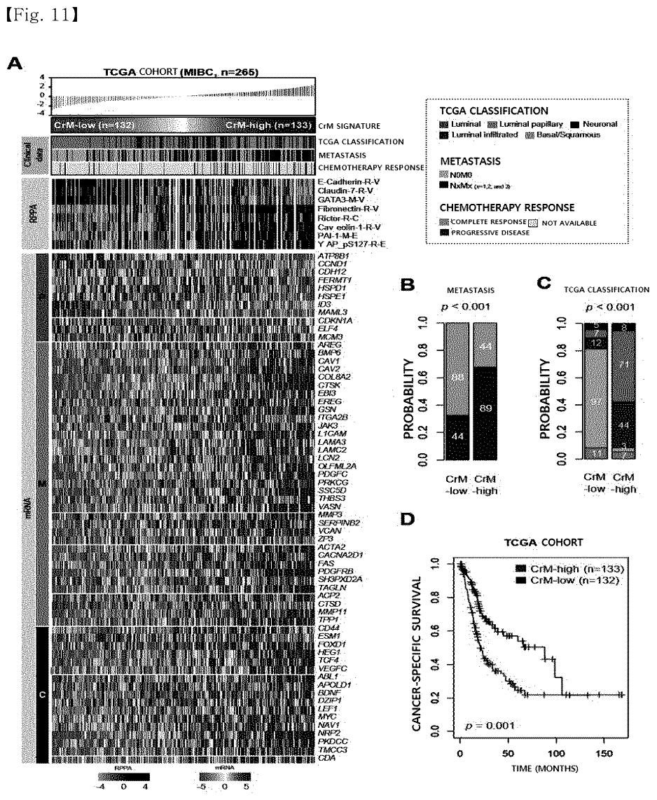

A-D of FIG. 11 are diagrams of gene expression patterns and survival analysis for MIBC patients in a TCGA cohort based on the CrM signature of the present disclosure.

A of FIG. 11: Heatmap of TCGA cohort divided into two groups based on CrM signature

B of FIG. 11: Comparison of metastasis states of two groups stratified by CrM signature

C of FIG. 11: TCGA classification distribution of two groups stratified by CrM signature

D of FIG. 11: Kaplan-Meier plot of two groups in TCGA cohort stratified by CrM signature

### BEST MODE OF THE INVENTION

Hereinafter, exemplary embodiments of the present disclosure will be described in detail with reference to the accompanying drawings. In the following description, detailed descriptions of techniques well-known to those skilled in the field may be omitted. Further, in describing the present disclosure, the detailed description of associated known functions or constitutions will be omitted if it is determined to unnecessarily make the gist of the present disclosure unclear. Terminologies used herein are terminologies used to properly express preferred embodiments of the present disclosure, which may vary according to a user, an operator's intention, or customs in the field to which the present disclosure pertains.

Accordingly, definitions of the terminologies need to be described based on contents throughout this specification. Throughout the specification, unless explicitly described to the contrary, when a certain part “comprises” a certain component, it will be understood to imply the inclusion of stated elements but not the exclusion of any other elements.

The present disclosure provides a biomarker of metastatic cancer including a gene selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

As used herein, “polynucleotide (or nucleotide, nucleic acid)” has a meaning comprehensively including DNA (gDNA and cDNA) and RNA molecules, and nucleotides, which are basic structural units in nucleic acid molecules, include not only natural nucleotides but also analogues with modified sugar or base sites.

As used herein, “polypeptide (or protein)” is interpreted to include an amino acid sequence that exhibits substantial identity to the corresponding amino acid sequence. The substantial identity means an amino acid sequence having at least 60% homology, more preferably at least 80% homology, and most preferably at least 90% homology when aligning any other sequence to match the amino acid sequence of the present disclosure as much as possible and analyzing the aligned sequence using an algorithm commonly used in the art, but is not limited thereto. In general, higher % identity is more preferable. In addition, the polypeptide having identity includes a polypeptide associated with a beta-adipate pathway while including an amino acid sequence in which one or more amino acid residues are deleted, substituted, inserted, and/or added in a polypeptide having a specific amino acid sequence described. In general, it is more preferable that the number of deletions, substitutions, insertions, and/or additions is fewer.

The polynucleotide of the present disclosure is not limited to nucleic acid molecules encoding the described specific amino acid sequence (polypeptide), and is interpreted to include a nucleic acid molecule encoding an amino acid sequence exhibiting substantial identity to a specific amino acid sequence or a polypeptide having a corresponding function thereto as described above. The substantial identity means an amino acid sequence having at least 60% homology, more preferably at least 80% homology, and most preferably at least 90% homology when aligning any other sequence to match the amino acid sequence of the present disclosure as much as possible and analyzing the aligned sequence using an algorithm commonly used in the art, but is not limited thereto.

The polypeptide having the corresponding function includes, for example, polypeptides having an amino acid sequence in which one or more amino acids are deleted, substituted, inserted, and/or added. These polypeptides include polypeptides consisting of amino acid sequences in which one or more amino acid residues are deleted, substituted, inserted, and/or added as described above, and associated with 3-hydroxypropionic acid synthesis, and preferably have a small number of deletions, substitutions, insertions, and/or additions of amino acid residues. In addition, the polypeptides include polypeptides having an amino acid sequence having at least about 60% identity with the specific amino acid sequence described above and functioning as a biomarker for diagnosing or prognosing bladder cancer, and preferably have higher identity.

As used herein, the term “complementary” or “complementarity” refers to the capacity of forming a double-stranded polynucleotide by binding purine and pyrimidine nucleotides via hydrogen bonds, and includes partially complementary cases. The following base pairs are related with complementarity: guanine and cytosine; adenine and thymine; and adenine and uracil. The “complementary” means that the aforementioned relationship is substantially applied to all base pairs including two single-stranded polynucleotides throughout the full-length molecule. The “partially complementary” refers to a relationship in which one of the two single-stranded polynucleotides is shorter in length, so that some of one of the molecules remains single-stranded.

According to an embodiment of the present disclosure, when the expression of the gene is increased compared to a reference value of a control group, the infiltration (invasion) or migration of cancer cells may be increased.

According to an embodiment of the present disclosure, the ATP8B1 includes a base sequence of SEQ ID NO: 1;
CCND1 includes a base sequence of SEQ ID NO: 2;
CDH12 includes a base sequence of SEQ ID NO: 3;
FERMT1 includes a base sequence of SEQ ID NO: 4;
HSPD1 includes a base sequence of SEQ ID NO: 5;
HSPE1 includes a base sequence of SEQ ID NO: 6;
ID3 includes a base sequence of SEQ ID NO: 7;
MAML3 includes a base sequence of SEQ ID NO: 8;
CDKN1A includes a base sequence of SEQ ID NO: 9;
ELF4 includes a base sequence of SEQ ID NO: 10;
MCM3 includes a base sequence of SEQ ID NO: 11;
AREG includes a base sequence of SEQ ID NO: 12;
BMP6 includes a base sequence of SEQ ID NO: 13;
CAV1 includes a base sequence of SEQ ID NO: 14;
CAV2 includes a base sequence of SEQ ID NO: 15;
COL8A2 includes a base sequence of SEQ ID NO: 16;
CTSK includes a base sequence of SEQ ID NO: 17;
EBI3 includes a base sequence of SEQ ID NO: 18;
EREG includes a base sequence of SEQ ID NO: 19;
GSN includes a base sequence of SEQ ID NO: 20;
ITGA2B includes a base sequence of SEQ ID NO: 21;
JAK3 includes a base sequence of SEQ ID NO: 22;
L1CAM includes a base sequence of SEQ ID NO: 23;
LAMA43 includes a base sequence of SEQ ID NO: 24;
LAMC2 includes a base sequence of SEQ ID NO: 25;
LCN2 includes a base sequence of SEQ ID NO: 26;
OLFML2A includes a base sequence of SEQ ID NO: 27;
PDGFC includes a base sequence of SEQ ID NO: 28;
PRKCG includes a base sequence of SEQ ID NO: 29;
SSCSD includes a base sequence of SEQ ID NO: 30;
THBS3 includes a base sequence of SEQ ID NO: 31;
VASN includes a base sequence of SEQ ID NO: 32;
MMP3 includes a base sequence of SEQ ID NO: 33;
SERPINB2 includes a base sequence of SEQ ID NO: 34;
VCAN includes a base sequence of SEQ ID NO: 35;
ZP3 includes a base sequence of SEQ ID NO: 36;
ACTA2 includes a base sequence of SEQ ID NO: 37;
CACNA2D1 includes a base sequence of SEQ ID NO: 38;
FAS includes a base sequence of SEQ ID NO: 39;
PDGFRB includes a base sequence of SEQ ID NO: 40;
SH3PXD2A includes a base sequence of SEQ ID NO: 41;
TAGLN includes a base sequence of SEQ ID NO: 42;
ACP2 includes a base sequence of SEQ ID NO: 43;
CTSD includes a base sequence of SEQ ID NO: 44;
MMP11 includes a base sequence of SEQ ID NO: 45;
TPP1 includes a base sequence of SEQ ID NO: 46;
CD44 includes a base sequence of SEQ ID NO: 47;
ESM1 includes a base sequence of SEQ ID NO: 48;
FOXD1 includes a base sequence of SEQ ID NO: 49;
HEG1 includes a base sequence of SEQ ID NO: 50;
TCF4 includes a base sequence of SEQ ID NO: 51;
VEGFC includes a base sequence of SEQ ID NO: 52;
ABL1 includes a base sequence of SEQ ID NO: 53;
APOLD1 includes a base sequence of SEQ ID NO: 54;
BDNF includes a base sequence of SEQ ID NO: 55;
DZIP1 includes a base sequence of SEQ ID NO: 56;
LEF1 includes a base sequence of SEQ ID NO: 57;
MYC includes a base sequence of SEQ ID NO: 58;
NAV1 includes a base sequence of SEQ ID NO: 59;
NRP2 includes a base sequence of SEQ ID NO: 60;
PKDCC includes a base sequence of SEQ ID NO: 61;
TMCC3 includes a base sequence of SEQ ID NO: 62; and
CDA includes a base sequence of SEQ ID NO: 63.

According to an embodiment of the present invention, when the expression of the gene is increased compared to the reference value of the control group, metastasis of cancer cells may increase.

According to an embodiment of the present disclosure, if the expression of the gene increases compared to the reference value of the control group, poor prognosis may be exhibited, and the poor prognosis may be reduced metastasis of cancer or viability of a subject.

According to an embodiment of the present disclosure, the cancer may be selected from the group consisting of bladder cancer, stomach cancer, colon cancer, rectal cancer, anal cancer, bone cancer, cerebrospinal tumor, head and neck cancer, thymoma, mesothelioma, esophageal cancer, biliary tract cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer, preferably bladder cancer, but is not limited thereto.

The “bladder cancer” of the present disclosure refers to a malignant tumor occurring in the bladder. Most cancers occurring in the bladder are epithelial tumors derived from epithelial cells, and malignant epithelial tumors include urothelial carcinoma, squamous cell carcinoma, and urothelial adenocarcinoma. Depending on the stage of progression, bladder cancer is classified into non-muscle-invasive bladder cancer (NMIBC), which is limited only to the bladder mucosa or submucosa, muscle-invasive bladder cancer (MIBC), which invades the muscle layer, and metastatic bladder cancer. In addition, most bladder cancers are epithelial tumors derived from epithelial cells, and malignant epithelial tumors include transitional cell carcinoma, squamous cell carcinoma, and adenocarcinoma. Other types of bladder cancer include sarcoma derived from bladder muscle, small cell carcinoma derived from nerve cells, malignant lymphoma, and metastatic bladder cancer that has spread from other organs to the bladder.

Further, the present disclosure provides a biomarker composition for predicting metastasis or prognosis of cancer, including a preparation for measuring the expression level of a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

In the present disclosure, expression measurement (or detection) includes quantitative and/or qualitative analysis, including detection of presence or absence and detection of expression amount. Such methods are known in the field, and those skilled in the field may select an appropriate method for implementing the present disclosure.

In the present disclosure, the material for measuring the expression level of the gene may be a material for detecting at least one of the presence, amount, and presence pattern of mRNA transcribed by the gene, a protein encoded by the gene, or both. The present disclosure may be used for diagnosing or prognosing cancer through quantitative and/or qualitative detection of the gene at a nucleic acid level, particularly at an mRNA level.

In the present disclosure, a material for measuring the expression level of the gene may be at least one of a primer, a probe, an aptamer, and an antisense that specifically binds to at least one selected from the group consisting of a nucleotide sequence of the gene, a sequence complementary thereto, a fragment of the nucleotide, and a sequence complementary thereto.

For example, in order to measure the presence, the amount or the pattern of mRNA of the gene by RT-PCR, a probe and/or primer pair specific for mRNA of the gene is included. The primer or probe refers to a nucleic acid sequence having a free 3′ hydroxyl group that may bind complementarily to a template and allow reverse transcriptase or DNA polymerase to initiate replication of the template. The material for measuring the gene expression used herein may be labeled with a color-emitting, luminescent or fluorescent material for signal detection. For example, Northern blot or reverse transcriptional polymerase chain reaction (PCR) is used to detect mRNA. The latter case is a method capable of determining the presence/absence or expression level of a specific gene by isolating RNA of a specimen, particularly mRNA, synthesizing cDNA from the isolated RNA, and then detecting a specific gene in the specimen using a specific primer or a combination of a primer and a probe.

In the present disclosure, the material for measuring the expression level of the gene may be at least one selected from an oligopeptide, a monoclonal antibody, a polyclonal antibody, a chimeric antibody, an antibody fragment, a ligand, a peptide nucleic acid (PNA), an aptamer, an avidity multimer, and a peptidomimetics, that specifically binds to at least one of a polypeptide encoded in the nucleotide sequence, a polypeptide encoded in the complementary sequence, and a polypeptide encoded in a fragment of the nucleotide sequence.

In the present disclosure, the material for measuring the expression level of the gene may be a material for detecting at least one of the presence, amount, and presence pattern of a conjugate of a protein encoded by the gene of the present disclosure and messenger RNA (mRNA) transcribed by the gene.

In the present disclosure, the material for measuring the expression level of the gene may include materials used in various gene (biomarker) detection methods known in the art. For example, the material for measuring the expression level of the gene may be a detection reagent for measuring the gene expression by at least one method selected from the group consisting of reverse transcription polymerase chain reaction, competitive polymerase chain reaction, real-time polymerase chain reaction, nuclease protection assay (RNase, S1 nuclease assay), in situ hybridization method, DNA microarray using method, Northern blot, Western blot, Enzyme Linked Immuno Sorbent Assay (ELISA), radioimmunoassay, immunodiffusion assay, immunoelectrophoresis, tissue immunostaining, immunoprecipitation assay, complement fixation assay, FACS, mass spectrometry, and protein microarray using method, but is not limited thereto.

The present disclosure measures the expression of the corresponding gene using various quantitative and/or qualitative analysis methods for nucleic acids and/or proteins known in the field. For example, for detection at the RNA level and detection of the expression level or pattern, methods using reverse transcription polymerase chain reaction (RT-PCR)/polymerase chain reaction, competitive RT-PCR, real-time RT-PCR, nuclease protection assay (NPA) such as RNase, S1 nuclease assay, in situ hybridization, DNA microarray or chip or northern blot, etc. may be used, and these analysis methods are known and may be performed using commercially available kits, and those skilled in the field may select an appropriate method for implementing the present disclosure.

According to an embodiment of the present disclosure, the preparation for measuring the expression level of the gene may include a primer pair selected from the group consisting of a primer pair of SEQ ID NOs: 64 and 65; a primer pair of SEQ ID NOs: 66 and 67; a primer pair of SEQ ID NOs: 68 and 69; a primer pair of SEQ ID NOs: 70 and 71; a primer pair of SEQ ID NOs: 72 and 73; a primer pair of SEQ ID NOs: 74 and 75; a primer pair of SEQ ID NOs: 76 and 77; a primer pair of SEQ ID NOs: 78 and 79; a primer pair of SEQ ID NOs: 80 and 81; a primer pair of SEQ ID NOs: 82 and 83; a primer pair of SEQ ID NOs: 84 and 85; a primer pair of SEQ ID NOs: 86 and 87; a primer pair of SEQ ID NOs: 88 and 89; a primer pair of SEQ ID NOs: 90 and 91; a primer pair of SEQ ID NOs: 92 and 93; a primer pair of SEQ ID NOs: 94 and 95; a primer pair of SEQ ID NOs: 96 and 97; a primer pair of SEQ ID NOs: 98 and 99; a primer pair of SEQ ID NOs: 100 and 101; a primer pair of SEQ ID NOs: 102 and 103; a primer pair of SEQ ID NOs: 104 and 105; a primer pair of SEQ ID NOs: 106 and 107; a primer pair of SEQ ID NOs: 108 and 109; a primer pair of SEQ ID NOs: 110 and 111; a primer pair of SEQ ID NOs: 112 and 113; a primer pair of SEQ ID NOs: 114 and 115; a primer pair of SEQ ID NOs: 116 and 117; a primer pair of SEQ ID NOs: 118 and 119; a primer pair of SEQ ID NOs: 120 and 121; a primer pair of SEQ ID NOs: 122 and 123; a primer pair of SEQ ID NOs: 124 and 125; a primer pair of SEQ ID NOs: 126 and 127; a primer pair of SEQ ID NOs: 128 and 129; a primer pair of SEQ ID NOs: 130 and 131; a primer pair of SEQ ID NOs: 132 and 133; a primer pair of SEQ ID NOs: 134 and 135; a primer pair of SEQ ID NOs: 136 and 137; a primer pair of SEQ ID NOs: 138 and 139; a primer pair of SEQ ID NOs: 140 and 141; a primer pair of SEQ ID NOs: 142 and 143; a primer pair of SEQ ID NOs: 144 and 145; a primer pair of SEQ ID NOs: 146 and 147; a primer pair of SEQ ID NOs: 148 and 149; a primer pair of SEQ ID NOs: 150 and 151; a primer pair of SEQ ID NOs: 152 and 153; a primer pair of SEQ ID NOs: 154 and 155; a primer pair of SEQ ID NOs: 156 and 157; a primer pair of SEQ ID NOs: 158 and 159; a primer pair of SEQ ID NOs: 160 and 161; a primer pair of SEQ ID NOs: 162 and 163; a primer pair of SEQ ID NOs: 164 and 165; a primer pair of SEQ ID NOs: 166 and 167; a primer pair of SEQ ID NOs: 168 and 169; a primer pair of SEQ ID NOs: 170 and 171; a primer pair of SEQ ID NOs: 172 and 173; a primer pair of SEQ ID NOs: 174 and 175; a primer pair of SEQ ID NOs: 176 and 177; a primer pair of SEQ ID NOs: 178 and 179; a primer pair of SEQ ID NOs: 180 and 181; a primer pair of SEQ ID NOs: 182 and 183; a primer pair of SEQ ID NOs: 184 and 185; a primer pair of SEQ ID NOs: 186 and 187; and a primer pair of SEQ ID NOs: 188 and 189.

According to an embodiment of the present disclosure, the measuring of the expression level of the gene may be measuring the amount of mRNA transcribed by the gene or the amount of protein encoded by the gene.

Further, the present disclosure provides a kit for predicting metastasis or prognosis of cancer including the composition.

Further, the present disclosure provides a method of providing information for predicting metastasis or prognosis of cancer including: confirming the expression level of a gene selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA; and
comparing the expression level of the gene with a reference value of a control group.

As used herein, “tissue or cell sample” means an aggregation of similar cells obtained from a tissue of a subject or patient. The sources of the tissue or cell sample may be fresh, frozen and/or preserved organ or tissue samples or solid tissue from a biopsy or aspirate; blood or any blood component; and cells at any time point of pregnancy or development in a subject. The tissue sample may also be primary or cultured cells or cell lines.

The sample refers to a material or a mixture of materials containing one or more components capable of detecting a biomarker, and includes cells, tissues or body fluids derived from a living organism, particularly a human, such as whole blood, urine, plasma and serum, but is not limited thereto. The sample also includes cells or tissues cultured in vitro as well as cells or tissues derived directly from living organisms. Various samples may be used for the detection of the bladder cancer biomarker according to the present disclosure, but are not limited thereto. In an embodiment, urine, whole blood, serum and/or plasma may be used. In another embodiment, liver tissue/cells or in vitro cell cultures obtained from a living organism that has, is suspected of having, or is likely to have bladder cancer may be used, but are not limited thereto. In addition, the samples include fractions or derivatives of the blood, cells or tissues. In the case of using cells or tissues, cells themselves or lysates of the cells or tissues may be used.

In the method for predicting metastasis or prognosis of bladder cancer according to the present disclosure, the expression level of the gene may be measured using various known quantitative and/or qualitative measurement methods for nucleic acids and/or proteins as described above.

Various methods known in the field may be used to compare marker profiles between a control group and test groups using samples. For example, the methods may refer to comparison of digital images of expression profiles and comparison using a DB for expression data. The profile obtained through marker detection according to the present disclosure may be processed using a known data analysis method. For example, nearest neighbor classifier, partial-least squares, SVM, AdaBoost, and clustering-based classification methods may be used. In addition, various statistical processing methods may be used to confirm the significance of the method for diagnosing and prognosing bladder cancer according to the present disclosure. In addition, statistical processing may be used to determine the confidence level for a significant difference between the test material and the control group for predicting metastasis or prognosis of cancer. The raw data used for statistical processing are values analyzed doubly, triply, or multiply for each marker. These statistical analysis methods are very useful for making clinically significant judgments through statistical processing of clinical and genetic data as well as biomarkers.

According to an embodiment of the present disclosure, the biological sample may be at least one selected from the group consisting of blood, hair, saliva, epidermis, semen, vaginal exudate, isolated cells, tissue samples, dandruff, and bones.

According to an embodiment of the present disclosure, the expression level of the gene may be measured by a method selected from the group consisting of reverse transcriptase-polymerase chain reaction, real time-polymerase chain reaction, Western blot, Northern blot, enzyme linked immunosorbent assay (ELISA), radioimmunoassay (RIA), radioimmunodiffusion, and immunoprecipitation assay.

Further, the present disclosure provides a biomarker of cancer having drug resistance to gemcitabine including a gene selected from the group consisting of ATP8BJ, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

According to an embodiment of the present invention, the gene may increase drug resistance of cancer cells to gemcitabine.

Further, the present disclosure provides a biomarker composition for diagnosis of cancer having drug resistance to gemcitabine, including a preparation for measuring the expression level of a gene selected from the group consisting of ATP8B1, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

### MODES OF THE INVENTION

Hereinafter, the present disclosure will be described in more detail through Examples. These Examples are to explain the present disclosure in more detail, and it will be apparent to those skilled in the field that the scope of the present disclosure is not limited to these Examples.

### <Example 1> Preparation for Discovery of Biomarkers Associated with Bladder Cancer Metastasis

### <1-1> Cell Culture

Cell lines of the present disclosure were cultured and used in subsequent experiments. Specifically, a 5637 cell line as a human bladder cancer cell line was purchased from the American Type Culture Collection. Thereafter, a parental cell line and gemcitabine-resistant bladder cancer (GRC) cell lines were cultured in an RPMI 1640 medium containing 10% fetal bovine serum (FBS) and 1% penicillin/streptomycin. All of the cell lines were cultured under conditions of 5% CO₂ and 37° C.

### <1-2> Construction of Gemcitabine-Resistant Bladder Cancer Cell Lines (GRCs)

To analyze stepwise molecular mechanisms occurring during a process of anticancer drug resistance in bladder cancer patients, bladder cancer cell lines having resistance to gemcitabine (GEM) as a representative anticancer drug for bladder cancer were constructed. Specifically, five candidate groups A to E of the 5637 cell line were treated with 5 μM GEM (Eli Lilly and company, IN, USA) and cultured for 3 days. Thereafter, the residual cells were washed with phosphate-buffered saline (PBS) and replaced with a new medium. The process was repeated until the cells recovered to 90% confluency, and after each stage was completed, several cell stocks in the same state were stored in liquid nitrogen. Thereafter, the recovered cells were inoculated in a plate and additionally treated with GEM. A total of 15-phase GEM-resistant bladder cancer cell lines were constructed using the method, which were named GRCA, GRCB, GRCC, GRCD, and GRCE according to each group.

### <1-3> Cell Proliferation Analysis

For cell proliferation analysis, 1×10³ cells were seeded in a 96-well plate and cultured for 24 hours. Thereafter, the cells were treated with 1 μM GEM for 0, 24, 48, and 72 hours, and cell viability was measured at each time point. The cells were then treated with thiazolyl blue tetrazolium bromide (MTT) (Sigma-Aldrich, St. Louis, MO, USA) and reacted for 1 hour. After the reaction was completed, the medium containing MTT was fully removed, and MTT formazan was dissolved using dimethyl sulfoxide (DMSO, Duchefa Biochemie, BH Haarlem, Netherlands). Thereafter, the absorbance was measured at 540 nm using a spectrophotometer microplate reader (Victor 3).

In addition, for clonogenic assay, the cells were inoculated in a 12-well plate (500 cells/well for a GEM untreated group, 4000 cells/well for a GEM treated group) and cultured for 7 days under conditions of 5% CO₂ and 37° C., and then the cells were stained with 0.5% crystal violet and confirmed. The number of colonies formed at this time was calculated using the Image J program (NIH; National Institutes of Health, Bethesda, MD, USA).

### <1-4> Soft Agar Sphere Formation Analysis

A dissolved 1.4% noble agar solution was added in a 6-well plate, and agar was solidified at room temperature. GRC cell lines treated with 1 μM of GEM for 24 hours were washed with PBS and treated with trypsin. Thereafter, a dissolved 0.7% agar solution and a cell suspension were mixed in a 1:1 ratio and inoculated onto the solidified 1.4% noble agar. The added 0.7% agar was solidified, and then a cell culture medium was added to the plate. Thereafter, the cells were cultured for two weeks, and the formed spheres were analyzed under a microscope.

### <1-5> Cell Motility Analysis

Cell infiltration (invasion) and migration capacity were analyzed using the Boyden chamber assay. Specifically, a membrane with 8 μm pores was coated with matrigel (BD biosciences, Franklin Lakes, NJ, USA) for infiltration analysis. Additionally, the membrane was coated with type I collagen for motility analysis. Thereafter, based on each coated membrane, cells suspended in a serum-free medium were inoculated in an upper chamber, and a medium supplemented with 1% FBS for infiltration (invasion) analysis or 10% FBS for migration analysis was added to a bottom chamber. After culturing for 24 hours, the cells that had migrated to the opposite side of the membrane were fixed and stained using a DiffQuik staining solution (Sysmex, Kobe, Japan). Thereafter, the stained cells were analyzed for infiltration (invasion) and migration capacity using the Image J program (NIH, Bethesda, MD, USA).

In addition, for wound healing assay, the cells were inoculated at 3×10⁵ per well in a 6-well plate and cultured for 24 hours. Thereafter, a wound was made across the plate using a sterilized P200 pipette tip, and the cells were cultured for 24 hours. After culture, the degree of wound closure was calculated using the Image J program.

### <1-6> Motility Analysis of Gemcitabine-Resistant Bladder Cancer Cell Lines (3D Microfluidic System)

For motility analysis, a microfluidic device was fabricated. After fabrication, a poly-dimethylsiloxane (PDMS) (Sylgard 184, Dow Corning, USA) pre-polymer was poured into a Si master mold with a thickness of approximately 5 to 6 mm and heated at 80° C. for 2 hours. Thereafter, a start portion of a microchannel was punched to make an inlet hole. The PDMS layer was attached to sterilized cover glass, and a channel surface was coated with poly-D-lysinehydrobromide (PDL; Sigma-Aldrich, MO, USA) for 4 hours to increase the electrostatic interaction of collagen with the channel surface. Thereafter, the channel was washed three times with distilled water. The microfluidic device was then completely dried at 80° C.

A Type 1 collagen (Corning, New York, USA) solution (2 mg/ml) was added to a central channel of the prepared microfluidic device as a scaffold material and placed in an incubator at 37° C. for 30 minutes to form a gel. Thereafter, a collagen solution (35 μg/ml) was added to the medium channel to increase cell adhesion to the device surface and cultured for 30 minutes, and after the culture was completed, the channel was washed with a new medium. Thereafter, at each stage of P0, P3, P7, and P15, a suspension (2×10⁶ cells/ml) of the 5637GRCA cell line was inoculated into the medium channel and cultured for 30 minutes. The cells were cultured in the device for 3 days while replacing the medium daily, and the migration capacity of 5637GRCA cells was confirmed on day 3 of culture. The cells were fixed with 4% paraformaldehyde for 30 minutes at room temperature and permeabilized with 0.1% Triton X-100 for 10 minutes at room temperature. Thereafter, actin filaments were stained with phalloidin-594 (1:400, #A12381, Invitrogen, Carlsbad, CA, USA) for 2 hours. Nuclei were stained with Hoechst 33342 (1:2000, #62249, Thermo Fisher Scientific, Waltham, MA, USA) for 10 minutes. Thereafter, images were captured using a high-content screening microscope (CELENA X, Logos Biosystems, Korea) for image analysis. For three different devices, eight region of interest (ROI) images were obtained, and for migration images of cancer cells, an infiltration area (mm²), an average infiltration distance (μm), and the number of infiltrating cells were quantitatively analyzed using the Image J program.

### <1-7> Mouse Xenograft

For a tumor xenograft experiment, a 5637GRCA cell line (5×10⁶ cells/PBS and 100 μl of matrigel suspension) was injected subcutaneously into the flank of a 6-week-old male BALB/c nude mouse (Orient bio, Korea). After 8 weeks, the mouse was sacrificed and then tumor tissues were obtained. The tumor volume was measured using the following Equation 1.

$$tumor volume [ {mm}^{3} ] = ( length [ mm ] \times {width}^{2} [ {mm}^{2} ] ) / 2 \qquad [ Equation 1 ]$$

In addition, for a tumor metastasis experiment, 4×10⁶ 5637GRCA cells suspended in 200 μl PBS were injected into the tail vein of the 6-week-old male BALB/c nude mouse. After 10 weeks, the mouse was sacrificed, lung tissue and metastatic tumor tissue were obtained, tumor burden was confirmed, and tissue sections were embedded in paraffin and identified by H&E, MMP2, MMP3, CAV1, and L1CAM staining.

### <1-8> RNA Extraction and RNA-Sequencing Data Processing

Total RNA was extracted from the 5637GRC cell line using a RNeasy Mini Kit (#74316, Qiagen, CA, USA) according to the manufacturer's procedure. The RNA quality was assessed by gel electrophoresis, and the RNA concentration was measured using a Nanodrop spectrophotometer (ND-1000, Thermo Fisher Scientific, Waltham, MA, USA). A library configuration for whole transcriptome sequencing was performed using a kit, and sequencing was performed using HiSeq2500 (Illumina, CA, USA) with paired-end reads (2×200 bp). Thereafter, reference genome sequence data of Homo sapiens were obtained from the Ensemble genome browser (assembly ID: GRCh38) and reference genome indexing and read mapping of samples were performed using STAR software (ver. 2.6.1a). In addition, a binary alignment map file generated using FeatureCounts (version 1.6.2) software was calculated. RNA sequencing data were calculated as CPM values, normalized using quantile normalization, transformed to log 2 values, and then identified around the median across genes and samples. Thereafter, a dataset was deposited as the data series accession number GSE210954 in the National Center for Biotechnology Information (NCBI) Gene Expression Omnibus (GEO) public database. Target sequences for amplifying the selected genes of the present disclosure were shown in Table 1 below, and primer sequences for PCR were shown in Table 2 below.

TABLE 1

| | | Target |
|---|---|---|
| Gene | Target sequence | size |
| ATP8B1 | CCTCTCAACACAACCACAACAAAATAGAGCAAGTTGATTTTAGCTGGAATACATA | 152 bp |
| (SEQ ID NO: 1) | TGCTGATGGGAAGCTTGCATTTTATGACCACTATCTTATTGAGCAAATCCAGTCA | |
| | GGGAAAGAGCCAGAAGTACGACAGTTCTTCTTCTTGCTCGCA | |
| CCND1 | AGGCGGAGGAGAACAAACAGATCATCCGCAAACACGCGCAGACCTTCGTTGCCCT | 92 bp |
| (SEQ ID NO: 2) | CTGTGCCACAGATGTGAAGTTCATTTCCAATCCGCCC | |
| CDH12 | CCTGAGGCTGCTATCAAACCAAATTTTACAGTTCGTGACTTCAGAAACAACACAG | 115 bp |
| (SEQ ID NO: 3) | CGGGGATTGAAACCCGAAGAAATGGATACAGCCGCAGGCAGCAAGAGTTGTATTT | |
| | CCTCC | |
| FERMT1 | ACCACGGTGTGCAAAAAGACACAAATCCAAACAGCTGGCCGCCCGGATCCTGGAG | 159 bp |
| (SEQ ID NO: 4) | GCGCACCAGAACGTGGCCCAGATGCCCCTGGTCGAAGCCAAGCTGCGGTTCATCC | |
| | AGGCGTGGCAGTCACTGCCTGAGTTTGGCCTCACCTACTACCTTGTCAG | |
| HSPD1 | GAATGAACGGCTTGCAAAACTTTCAGATGGAGTGGCTGTGCTGAAGGTTGGTGGG | 146 bp |
| (SEQ ID NO: 5) | ACAAGTGATGTTGAAGTGAATGAAAAGAAAGACAGAGTTACAGATGCCCTTAATG | |
| | CTACAAGAGCTGCTGTTGAAGAAGGCATTGTTTTGG | |
| HSPE1 | GAGAGATTCAACCAGTTAGCGTGAAAGTTGGAGATAAAGTTCTTCTCCCAGAATA | 126 bp |
| (SEQ ID NO: 6) | TGGAGGCACCAAAGTAGTTCTAGATGACAAGGATTATTTCCTATTTAGAGATGGT | |
| | GACATTCTTGGAAAGT | |
| ID3 | TCATCGACTACATTCTCGACCTGCAGGTAGTCCTGGCCGAGCCAGCCCCTGGACC | 122 bp |
| (SEQ ID NO: 7) | CCCTGATGGCCCCCACCTTCCCATCCAGACAGCCGAGCTCACTCCGGAACTTGTC | |
| | ATCTCCAACGAC | |
| MAML3 | TGCAGCAATCACATCTACCCCGGCAGCACCTCCAGCCACAGCGGAATCCATACCC | 177 bp |
| (SEQ ID NO: 8) | AGTGCAGCAGGTCAATCAGTTTCAAGGTTCTCCCCAGGATATAGCAGCCGTAAGA | |
| | AGCCAAGCAGCCCTCCAGAGCATGCGAACGTCACGGCTGATGGCACAGAACGCAG | |
| | GCATGATGGGAA | |
| CDKN1A | AACAAAGCTGCTGCAACCACAGGGATTTCTTCTGTTCAGGCGCCATGTCAGAACC | 159 bp |
| (SEQ ID NO: 9) | GGCTGGGGATGTCCGTCAGAACCCATGCGGCAGCAAGGCCTGCCGCCGCCTCTTC | |
| | GGCCCAGTGGACAGCGAGCAGCTGAGCCGCGACTGTGATGCGCTAATGG | |
| ELF4 | AAGACCAAGGGCAACCGAAGTACCTCACCTGTCACTGACCCCAGCATCCCCATTA | 123 bp |
| (SEQ ID NO: 10) | GGAAGAAATCAAAGGATGGCAAAGGCAGCACCATCTATCTGTGGGAGTTCCTCCT | |
| | GGCTCTTCTGCAA | |
| MCM3 | ACACTCCAAAGACGGCAGACTCACAGGAGACCAAGGAATCCCAGAAAGTGGAGTT | 103 bp |
| (SEQ ID NO: 11) | GAGTGAATCCAGGTTGAAGGCATTCAAGGTGGCCCTCTTGGATGTGTT | |
| AREG | CTGGATTGGACCTCAATGACACCTACTCTGGGAAGCGTGAACCATTTTCTGGGGA | 110 bp |
| (SEQ ID NO: 12) | CCACAGTGCTGATGGATTTGAGGTTACCTCAAGAAGTGAGATGTCTTCAGGGAGT | |
| BMP6 | GATCATTGCACCCAAGGGCTATGCTGCCAATTACTGTGATGGAGAATGCTCCTTC | 164 bp |
| (SEQ ID NO: 13) | CCACTCAACGCACACATGAATGCAACCAACCACGCGATTGTGCAGACCTTGGTTC | |
| | ACCTTATGAACCCCGAGTATGTCCCCAAACCGTGCTGTGCGCCAACTAAGCTAA | |
| CAV1 | TCAACCGCGACCCTAAACACCTCAACGATGACGTGGTCAAGATTGACTTTGAAGA | 112 bp |
| (SEQ ID NO: 14) | TGTGATTGCAGAACCAGAAGGGACACACAGTTTTGACGGCATTTGGAAGGCCAGC | |
| (SEQ ID NO: | TT | |
| CAV2 | GAATTCTCTTTGCCACCCTCAGCTGTCTGCACATCTGGATTTTAATGCCTTTTGT | 110 bp |
| (SEQ ID NO: 15) | AAAGACCTGCCTAATGGTTCTGCCTTCAGTGCAGACAATATGGAAGAGTGTGACA | |
| COL8A2 | ACAAGAAGGGCTACCTGGACCAGGCATCTGGTGGGGCCGTGCTCCAGCTGCGGCC | 139 bp |
| (SEQ ID NO: 16) | CAACGACCAGGTCTGGGTGCAGATGCCGTCGGACCAGGCCAACGGCCTCTACTCC | |
| | ACGGAGTACATCCACTCCTCCTTTTCAGG | |
| CTSK | TTCCCGCAGTAATGACACCCTTTATATCCCAGAATGGGAAGGTAGAGCCCCAGAC | 139 bp |
| (SEQ ID NO: 17) | TCTGTCGACTATCGAAAGAAAGGATATGTTACTCCTGTCAAAAATCAGGGTCAGT | |
| | GTGGTTCCTGTTGGGCTTTTAGCTCTGTG | |
| EBI3 | GATCCGTTACAAGCGTCAGGGAGCTGCGCGCTTCCACCGGGTGGGGCCCATTGAA | 157 bp |
| (SEQ ID NO: 18) | GCCACGTCCTTCATCCTCAGGGCTGTGCGGCCCCGAGCCAGGTACTACGTCCAAG | |
| | TGGCGGCTCAGGACCTCACAGACTACGGGGAACTGAGTGACTGGAGT | |
| EREG | GGACATGAGTCAAAACTACTGCAGGTGTGAAGTGGGTTATACTGGTGTCCGATGT | 150 bp |
| (SEQ ID NO: 19) | GAACACTTCTTTTTAACCGTCCACCAACCTTTAAGCAAAGAATATGTGGCTTTGA | |
| | CCGTGATTCTTATTATTTTGTTTCTTATCACAGTCGTCGG | |
| GSN | AGCTGGATGACTACCTGAACGGCCGGGCCGTGCAGCACCGTGAGGTCCAGGGCTT | 132 bp |
| (SEQ ID NO: 20) | CGAGTCGGCCACCTTCCTAGGCTACTTCAAGTCTGGCCTGAAGTACAAGAAAGGA | |
| | GGTGTGGCATCAGGATTCAAGC | |
| ITGA2B | TGACAACGGATACCCAGACCTGATCGTGGGAGCTTACGGGGCCAACCAGGTGGCT | 141 bp |
| (SEQ ID NO: 21) | GTGTACAGAGCTCAGCCAGTGGTGAAGGCCTCTGTCCAGCTACTGGTGCAAGATT | |
| | CACTGAATCCTGCTGTGAAGAGCTGTGTCCT | |
| JAK3 | TCAGCCCCAATCCCAATACCAGCTGAGTCAGATGACATTTCACAAGATCCCTGCT | 174 bp |
| (SEQ ID NO: 22) | GACAGCCTGGAGTGGCATGAGAACCTGGGCCATGGGTCCTTCACCAAGATTTACC | |
| | GGGGCTGTCGCCATGAGGTGGTGGATGGGGAGGCCCGAAAGACAGAGGTGCTGCT | |
| | GAAGGTCAT | |
| L1CAM | ATGCCTACATCTACGTTGTCCAGCTGCCAGCCAAGATCCTGACTGCGGACAATCA | 157 bp |
| (SEQ ID NO: 23) | GACGTACATGGCTGTCCAGGGCAGCACTGCCTACCTTCTGTGCAAGGCCTTCGGA | |
| | GCGCCTGTGCCCAGTGTTCAGTGGCTGGACGAGGATGGGACAACAGT | |
| LAMA3 | CTTACCACCTACTGACCACCTCCAGGCCTCATTTGGATTTCAGACCTTTCAACCC | 118 bp |
| (SEQ ID NO: 24) | AGTGGCATATTATTAGATCATCAGACATGGACAAGGAACCTGCAGGTCACTCTGG | |
| | AAGATGGT | |
| LAMC2 | TCACTCTCAAGCCTGGTAACCAGGCATATGGATGAGTTCAAGCGTACACAGAAGA | 117 bp |
| (SEQ ID NO: 25) | ATCTGGGAAACTGGAAAGAAGAAGCACAGCAGCTCTTACAGAATGGAAAAAGTGG | |
| | GAGAGAG | |
| LCN2 | AGACAAAGACCCGCAAAAGATGTATGCCACCATCTATGAGCTGAAAGAAGACAAG | 83 bp |
| (SEQ ID NO: 26) | AGCTACAATGTCACCTCCGTCCTGTTTA | |
| OLFML2A | GACATCAGCAAGTATGGCAGTGTGCAGAAAAGCTTTGCAGACAGAGGCCTCCCAA | 101 bp |
| (SEQ ID NO: 27) | AACCTCCCAAGGAGAAGCTGCTTCAGGTGGAGAAGCTGAGAAAGGA | |
| PDGFC | GCACACCTCGTAACTTCTCAGTGTCCATAAGGGAAGAACTAAAGAGAACCGATAC | 96 bp |
| (SEQ ID NO: 28) | CATTTTCTGGCCAGGTTGTCTCCTGGTTAAACGCTGTGGTG | |
| PRKCG | CGAAGTCAAGAGCCACAAGTTCACCGCTCGCTTCTTCAAGCAGCCCACCTTCTGC | 89 bp |
| (SEQ ID NO: 29) | AGCCACTGCACCGACTTCATCTGGGGTATCGGAA | |
| SSC5D | ACTCCATCTCAGACCCCTTCAGCTGGAGCTGGATTCCTGGACTGGGGAGAGATCG | 139 bp |
| (SEQ ID NO: 30) | GGATGCCTGGCTCCCGGGAGAGCTGGCCACCAAGCCCTCTGCAAGTGTGACTGCC | |
| | AGTGTTCTGGAGAAAACAACCACGAAGGC | |
| THBS3 | CCAACCCACTACAGACAGACAGGGATGAGGACGGGGTGGGAGATGCTTGCGACAG | 123 bp |
| (SEQ ID NO: 31) | CTGCCCTGAAATGAGCAATCCTACCCAGACAGATGCAGACAGCGACCTGGTGGGG | |
| | GATGTCTGTGATA | |
| VASN | TCTCACCTATCGCAACCTATCGGGCCCTGATAAGCGGCTGGTGACGCTGCGACTG | 131 bp |
| (SEQ ID NO: 32) | CCTGCCTCGCTCGCTGAGTACACGGTCACCCAGCTGCGGCCCAACGCCACTTACT | |
| | CCGTCTGTGTCATGCCTTTGG | |
| MMP3 | AGGCTTTCCCAAGCAAATAGCTGAAGACTTTCCAGGGATTGACTCAAAGATTGAT | 151 bp |
| (SEQ ID NO: 33) | GCTGTTTTTGAAGAATTTGGGTTCTTTTATTTCTTTACTGGATCTTCACAGTTGG | |
| | AGTTTGACCCAAATGCAAAGAAAGTGACACACACTTTGAAG | |
| SERPINB2 | ATTACTCCTCAGAACCCCAGGCAGTAGACTTCCTAGAATGTGCAGAAGAAGCTAG | 135 bp |
| (SEQ ID NO: 34) | AAAAAAGATTAATTCCTGGGTCAAGACTCAAACCAAAGGCAAAATCCCAAACTTG | |
| | TTACCTGAAGGTTCTGTAGATGGGG | |
| VCAN | GAATGTCACTCTAATCCCTGTCGTAATGGAGCCACTTGTGTTGATGGTTTTAACA | 140 bp |
| (SEQ ID NO: 35) | CATTCAGGTGCCTCTGCCTTCCAAGTTATGTTGGTGCACTTTGTGAGCAAGATAC | |
| | CGAGACATGTGACTATGGCTGGCACAAATT | |
| ZP3 | TTCCACTTTGCTAATGACTCCAGAAACATGATATACATCACCTGCCACCTGAAGG | 162 bp |
| (SEQ ID NO: 36) | TCACCCTAGCTGAGCAGGACCCAGATGAACTCAACAAGGCCTGTTCCTTCAGCAA | |
| | GCCTTCCAACAGCTGGTTCCCAGTGGAAGGCTCGGCTGACATCTGTCAATGC | |
| ACTA2 | GGGAATGGGACAAAAAGACAGCTACGTGGGTGACGAAGCACAGAGCAAAAGAGGA | 299 bp |
| (SEQ ID NO: 37) | ATCCTGACCCTGAAGTACCCGATAGAACATGGCATCATCACCAACTGGGACGACA | |
| | TGGAAAAGATCTGGCACCACTCTTTCTACAATGAGCTTCGTGTTGCCCCTGAAGA | |
| | GCATCCCACCCTGCTCACGGAGGCACCCCTGAACCCCAAGGCCAACCGGGAGAAA | |
| | ATGACTCAAATTATGTTTGAGACTTTCAATGTCCCAGCCATGTATGTGGCTATCC | |
| | AGGCGGTGCTGTCTCTCTATGCCT | |
| CACNA2D1 | CTGGCCTAGCTCGATATTATCCAGCTTCACCATGGGTTGATAATAGTAGAACTCC | 157 bp |
| (SEQ ID NO: 38) | AAATAAGATTGACCTTTATGATGTACGCAGAAGACCATGGTACATCCAAGGAGCT | |
| | GCATCTCCTAAAGACATGCTTATTCTGGTGGATGTGAGTGGAAGTGT | |
| FAS | GTATGTGAACACTGTGACCCTTGCACCAAATGTGAACATGGAATCATCAAGGAAT | 133 bp |
| (SEQ ID NO: 39) | GCACACTCACCAGCAACACCAAGTGCAAAGAGGAAGGATCCAGATCTAACTTGGG | |
| | GTGGCTTTGTCTTCTTCTTTTGC | |
| PDGFRB | AGCAGTTCTACAATGCCATCAAACGGGGTTACCGCATGGCCCAGCCTGCCCATGC | 166 bp |
| (SEQ ID NO: 40) | CTCCGACGAGATCTATGAGATCATGCAGAAGTGCTGGGAAGAGAAGTTTGAGATT | |
| | CGGCCCCCCTTCTCCCAGCTGGTGCTGCTTCTCGAGAGACTGTTGGGCGAAGGTT | |
| | A | |
| SH3PXD24 | TTCCAACTGCCAAAGCCACCAGAGCCCCCTTCTGTTGAGGTGGAGTACTACACCA | 122 bp |
| (SEQ ID NO: 41) | TTGCCGAATTCCAGTCGTGCATTTCCGATGGCATCAGCTTTCGGGGTGGACAGAA | |
| | GGCAGAGGTCAT | |
| TAGLN | CATGTTCCAGACTGTTGACCTCTTTGAAGGCAAAGACATGGCAGCAGTGCAGAGG | 143 bp |
| (SEQ ID NO: 42) | ACCCTGATGGCTTTGGGCAGCTTGGCAGTGACCAAGAATGATGGGCACTACCGTG | |
| | GAGATCCCAACTGGTTTATGAAGAAAGCGCAGG | |
| ACP2 | AACCTGACCCTAATGGCGACCACCTCCCAGCTCCCCAAGCTGCTGGTTTACTCTG | 139 bp |
| (SEQ ID NO: 43) | CGCACGACACTACCCTGGTTGCCCTGCAAATGGCACTGGATGTCTACAATGGTGA | |
| | ACAAGCCCCCTACGCCTCCTGCCACATAT | |
| CTSD | GCAAACTGCTGGACATCGCTTGCTGGATCCACCACAAGTACAACAGCGACAAGTC | 101 bp |
| (SEQ ID NO: 44) | CAGCACCTACGTGAAGAATGGTACCTCGTTTGACATCCACTATGGC | |
| MMP11 | CGACTATGATGAGACCTGGACTATCGGGGATGACCAGGGCACAGACCTGCTGCAG | 145 bp |
| (SEQ ID NO: 45) | GTGGCAGCCCATGAATTTGGCCACGTGCTGGGGCTGCAGCACACAACAGCAGCCA | |
| | AGGCCCTGATGTCGGCCTTCTACACCTTTCGCTAC | |
| TPP1 | GGTGGCTTCAGCAATGTGTTCCCACGGCCTTCATACCAGGAGGAAGCTGTAACGA | 99 bp |
| (SEQ ID NO: 46) | AGTTCCTGAGCTCTAGCCCCCACCTGCCACCATCCAGTTACTTC | |
| CD44 | AGCAACTGAGACAGCAACCAAGAGGCAAGAAACCTGGGATTGGTTTTCATGGTTG | 115 bp |
| ( [symbol] 47) | TTTCTACCATCAGAGTCAAAGAATCATCTTCACACAACAACACAAATGGCTGGTAC | |
| | GTCT | |
| ESM1 | TTCAGTAACCAAGTCTTCCAACAGATTTGTTTCTCTCACGGAGCATGACATGGCAT | 104 bp |
| (SEQ ID NO: 48) | CTGGAGATGGCAATATTGTGAGAGAAGAAGTTGTGAAAGAGAATGCTG | |
| FOXD1 | CTCGTATATCGCGCTCATCACTATGGCCATCCTGCAGAGCCCCAAGAAGCGGCTG | 173 bp |
| (SEQ ID NO: 49) | ACGCTGAGCGAGATCTGTGAGTTCATCAGCGGCCGCTTCCCCTACTACCGGGAGA | |
| | AGTTCCCCGCCTGGCAGAACAGCATCCGCCACAACCTCTCGCTCAACGACTGCTT | |
| | CGTCAAGA | |
| HEG1 | CCCAAAAATCCTCGCTCACAAGAATGGGGCCGAGAAGCTATTGAAATGCATGAGA | 143 bp |
| (SEQ ID NO: 50) | ATGGAAGTACCAAAAACCTCCTCCAGATGACGGATGTGTACTACTCGCCTACAAGT | |
| | GTAAGGAATCCAGAACTTGAACGAAACGGACT | |
| TCF4 | CAATCTCTCTCCACCTTTTGTCAATTCCAGAATACAAAGTAAAACAGAAAGGGGCTC | 137 bp |
| (SEQ ID NO: 51) | ATACTCATCTTATGGGAGAGAATCAAACTTACAGGGTTGCCACCAGCAGAGTCTCC | |
| | TTGGAGGTGACATGGATATGGGCA | |
| VEGFC | TCCACCACCAAACATGCAGCTGTTACAGACGGCCATGTACGAACCGCCAGAAGGC | 105 bp |
| (SEQ ID NO: 52) | TTGTGAGCCAGGATTTTCATATAGTGAAGAAGTGTGTCGTTGTGTCCCTT | |
| ABL1 | CCCTCCTTTGCTGAAATCCACCAAGCCTTTGAAACAATGTTCCAGGAATCCAGTAT | 78 bp |
| (SEQ ID NO: 53) | CTCAGACGAAGTGGAAAAGGAG | |
| APOLD1 | CAGATGCGAGAGATCCTGAGCTGCCTCGAGTTTTTCTGCCGCTGGCAGGGCTGCG | 135 bp |
| (SEQ ID NO: 54) | GGGACCGCCAGCTGCTGCAGTGCGGGAGGAACGCCTCCATCGCCCTGTACAATT | |
| | CTGTCTACTTCATCGTCTTCTTTGGC | |
| BDNF | CGTGATAGAAGAGCTGTTGGATGAGGACCAGAAAGTTCGGCCCAATGAAGAAAAC | 104 bp |
| (SEQ ID NO: 55) | AATAAGGACGCAGACTTGTACACGTCCAGGGTGATGCTCAGTAGTCAAG | |
| DZIP1 | GTCTTCAACTATTACGACCCCTCCTTTTAGTTCAGAGGAGGAGCAGGAGGACGAC | 145 bp |
| (SEQ ID NO: 56) | GACCTCATCCGGGCATACGCATCCCCAGGCCCACTTCCTGTGCCGCCACCACAAA | |
| | ACAAGGGCAGCTTCGGGAAGAACACAGTGAAAAGT | |
| LEF1 | ACCAGATTCTTGGCAGAAGGTGGCATGCCCTCTCCCGTGAAGAGCAGGCTAAATA | 116 bp |
| (SEQ ID NO: 57) | TTATGAATTAGCACGGAAAGAAAGACAGCTACATATGCAGCTTTATCCAGGCTGGT | |
| | CTGCA | |
| MYC | CCTGGTGCTCCATGAGGAGACACCGCCCACCACCAGCAGCGACTCTGAGGAGGA | 128 bp |
| (SEQ ID NO: 58) | ACAAGAAGATGAGGAAGAAATCGATGTTGTTTCTGTGGAAAAGAGGCAGGCTCCT | |
| | GGCAAAAGGTCAGAGTCTG | |
| NAV1 | TCAATGCCAACAAGGAAGAGCTGCTTCGGGTGCTCGACTGGGTACCCAAGCTGTG | 131 bp |
| (SEQ ID NO: 59) | GTATCATCTCCACACCTTCCTTGAGAAGCACAGCACCTCAGACTTCCTCATCGGCC | |
| | CTTGCTTCTTTCTGTCGTGT | |
| NRP2 | GATGATGAATACGAGGTGGACTGGAGCAATTCTTCTTCTGCAACCTCAGGGTCTG | 121 bp |
| (SEQ ID NO: 60) | GCGCCCCCTCGACCGACAAAGAAAAGAGCTGGCTGTACACCCTGGATCCCATCCT | |
| | CATCACCATCA | |
| PKDCC | AAGCAGCAGTACCGAGTACCAGTGTATCCCAGACAGCACCATCCCCCAGGAAGAC | 137 bp |
| (SEQ ID NO: 61) | TACCGCTGCTGGCCATCCTACCACCACGGGAGCTGCCTCCTTTCAGTGTTCAACC | |
| | TGGCTGAGGCTGTGGATGTCTGTGAGA | |
| TMCC3 | CCGTGAATGCTAAAGTTCTCCTGGGGAGGTGCATCAACGTGATCCTGGCCTTCAT | 79 bp |
| (SEQ ID NO: 62) | GACTGTCATCTTAGTGTGTGTGTC | |
| CDA | CTGAACGGACCGCTATCCAGAAGGCCGTCTCAGAAGGGTACAAGGATTTCAGGGC | 108 bp |
| (SEQ ID NO: 63) | AATTGCTATCGCCAGTGACATGCAAGATGATTTTATCTCTCCATGTGGGGCCT | |

TABLE 2

| Gene | SEQ ID NO: | Primer | Base sequence |
|---|---|---|---|
| ATP8B1 | SEQ ID NO: 64 | ATP8B1 qPCR-F | CCTCTCAACACAACCACAAC |
| | SEQ ID NO: 65 | ATP8B1 qPCR-R | TGCGAGCAAGAAGAAGAAC |
| CCND1 | SEQ ID NO: 66 | CCND1 qPCR F | AGGCGGAGGAGAACAAAC |
| | SEQ ID NO: 67 | CCND1 qPCR R | GGGCGGATTGGAAATGAAC |
| CDH12 | SEQ ID NO: 68 | CDH12 qPCR-F | CCTGAGGCTGCTATCAAAC |
| | SEQ ID NO: 69 | CDH12 qPCR-R | GGAGGAAATACAACTCTTGCTG |
| FERMT1 | SEQ ID NO: 70 | FERMT1 qPCR-F | ACCACGGTGTGCAAAAAG |
| | SEQ ID NO: 71 | FERMT1 qPCR-R | CTGACAAGGTAGTAGGTGAGG |
| HSPD1 | SEQ ID NO: 72 | HSPD1 qPCR-F | GAATGAACGGCTTGCAAAAC |
| | SEQ ID NO: 73 | HSPD1 qPCR-R | CCAAAACAATGCCTTCTTCAAC |
| HSPE1 | SEQ ID NO: 74 | HSPE1 qPCR-F | GAGAGATTCAACCAGTTAGCG |
| | SEQ ID NO: 75 | HSPE1 qPCR-R | ACTTTCCAAGAATGTCACCATC |
| ID3 | SEQ ID NO: 76 | ID3 qPCR-F | TCATCGACTACATTCTCGACC |
| | SEQ ID NO: 77 | ID3 qPCR-R | GTCGTTGGAGATGACAAGTTC |
| MAML3 | SEQ ID NO: 78 | MAML3 qPCR-F | TGCAGCAATCACATCTACCCC |
| | SEQ ID NO: 79 | MAML3 qPCR-R | TTCCCATCATGCCTGCGTTC |
| CDKN1A | SEQ ID NO: 80 | CDKN1A qPCR-F | AACAAAGCTGCTGCAACC |
| | SEQ ID NO: 81 | CDKN1A qPCR-R | CCATTAGCGCATCACAGTC |
| ELF4 | SEQ ID NO: 82 | ELF4 qPCR-F | AAGACCAAGGGCAACCGAAG |
| | SEQ ID NO: 83 | ELF4 qPCR-R | TTGCAGAAGAGCCAGGAGGAAC |
| MCM3 | SEQ ID NO: 84 | MCM3 qPCR-F | ACACTCCAAAGACGGCAGAC |
| | SEQ ID NO: 85 | MCM3 qPCR-R | AACACATCCAAGAGGGCCAC |
| AREG | SEQ ID NO: 86 | AREG qPCR-F | CTGGATTGGACCTCAATGAC |
| | SEQ ID NO: 87 | AREG qPCR-R | ACTCCCTGAAGACATCTCAC |
| BMP6 | SEQ ID NO: 88 | BMP6 qPCR-F | GATCATTGCACCCAAGGGCTAT |
| | SEQ ID NO: 89 | BMP6 qPCR-R | TTAGCTTAGTTGGCGCACAGCA |
| CAV1 | SEQ ID NO: 90 | CAV1 qPCR-F | TCAACCGCGACCCTAAACA |
| | SEQ ID NO: 91 | CAV1 qPCR-R | AAGCTGGCCTTCCAAATGC |
| CAV2 | SEQ ID NO: 92 | CAV2 qPCR-F | GAATTCTCTTTGCCACCCTC |
| | SEQ ID NO: 93 | CAV2 qPCR-R | TGTCACACTCTTCCATATTGTC |
| COL8A2 | SEQ ID NO: 94 | COL8A2 qPCR-F | ACAAGAAGGGCTACCTGGAC |
| | SEQ ID NO: 95 | COL8A2 qPCR-R | CCTGAAAAGGAGGAGTGGATG |
| CTSK | SEQ ID NO: 96 | CTSK qPCR-F | TTCCCGCAGTAATGACACC |
| | SEQ ID NO: 97 | CTSK qPCR-R | CACAGAGCTAAAAGCCCAAC |
| EBI3 | SEQ ID NO: 98 | EBI3 qPCR-F | GATCCGTTACAAGCGTCAG |
| | SEQ ID NO: 99 | EBI3 qPCR-R | ACTCCAGTCACTCAGTTCC |
| EREG | SEQ ID NO: 00 | EREG qPCR-F | GGACATGAGTCAAAACTACTGC |
| | SEQ ID NO: 01 | EREG qPCR-R | CCGACGACTGTGATAAGAAAC |
| GSN | SEQ ID NO: 02 | GSN qPCR-F | AGCTGGATGACTACCTGAAC |
| | SEQ ID NO: 03 | GSN qPCR-R | GCTTGAATCCTGATGCCAC |
| ITGA2B | SEQ ID NO: 04 | ITGA2B qPCR-F | TGACAACGGATACCCAGAC |
| | SEQ ID NO: 05 | ITGA2B qPCR-R | AGGACACAGCTCTTCACAG |
| JAK3 | SEQ ID NO: 06 | JAK3 qPCR-F | TCAGCCCCAATCCCAATACC |
| | SEQ ID NO: 07 | JAK3 qPCR-R | ATGACCTTCAGCAGCACCTC |
| L1CAM | SEQ ID NO: 08 | L1CAM qPCR-F | ATGCCTACATCTACGTTGTCC |
| | SEQ ID NO: 09 | L1CAM qPCR-R | ACTGTTGTCCCATCCTCGTC |
| LAMA3 | SEQ ID NO: 10 | LAMA3 qPCR-F | CTTACCACCTACTGACCACC |
| | SEQ ID NO: 11 | LAMA3 qPCR-R | ACCATCTTCCAGAGTGACC |
| LAMC2 | SEQ ID NO: 12 | LAMC2 qPCR-F | TCACTCTCAAGCCTGGTAAC |
| | SEQ ID NO: 13 | LAMC2 qPCR-R | CTCTCTCCCACTTTTTCCATTC |
| LCN2 | SEQ ID NO: 14 | LCN2 qPCR-F | AGACAAAGACCCGCAAAAG |
| | SEQ ID NO: 15 | LCN2 qPCR-R | TAAACAGGACGGAGGTGAC |
| OLFML2A | SEQ ID NO: 16 | OLFML2A qPCR-F | GACATCAGCAAGTATGGCAG |
| | SEQ ID NO: 17 | OLFML2A qPCR-R | TCCTTTCTCAGCTTCTCCAC |
| PDGFC | SEQ ID NO: 118 | PDGFC qPCR-F | GCACACCTCGTAACTTCTC |
| | SEQ ID NO: 119 | PDGFC qPCR-R | CACCACAGCGTTTAACCAG |
| PRKCG | SEQ ID NO: 120 | PRKCG qPCR-F | CGAAGTCAAGAGCCACAAG |
| | SEQ ID NO: 121 | PRKCG qPCR-R | TTCCGATACCCCAGATGAAG |
| SSC5D | SEQ ID NO: 122 | SSC5D qPCR-F | ACTCCATCTCAGACCCCTTC |
| | SEQ ID NO: 123 | SSC5D qPCR-R | GCCTTCGTGGTTGTTTTCTC |
| THBS3 | SEQ ID NO: 124 | THBS3 qPCR-F | CCAACCCACTACAGACAGAC |
| | SEQ ID NO: 125 | THBS3 qPCR-R | TATCACAGACATCCCCCACC |
| VASN | SEQ ID NO: 126 | VASN qPCR-F | TCTCACCTATCGCAACCTATC |
| | SEQ ID NO: 127 | VASN qPCR-R | CCAAAGGCATGACACAGAC |
| MMP3 | SEQ ID NO: 128 | MMP3 qPCR-F | AGGCTTTCCCAAGCAAATAG |
| | SEQ ID NO: 129 | MMP3 qPCR-R | CTTCAAAGTGTGTGTCACTTTC |
| SERPINB2 | SEQ ID NO: 130 | SERPINB2 qPCR-F | ATTACTCCTCAGAACCCCAG |
| | SEQ ID NO: 131 | SERPINB2 qPCR-R | CCCCATCTACAGAACCTTCAG |
| VCAN | SEQ ID NO: 132 | VCAN qPCR-F | GAATGTCACTCTAATCCCTGTC |
| | SEQ ID NO: 133 | VCAN qPCR-R | AATTTGTGCCAGCCATAGTC |
| ZP3 | SEQ ID NO: 134 | ZP3 qPCR-F | TTCCACTTTGCTAATGACTCC |
| | SEQ ID NO: 135 | ZP3 qPCR-R | GCATTGACAGATGTCAGCC |
| ACTA2 | SEQ ID NO: 136 | ACTA2 qPCR-F | GGGAATGGGACAAAAAGACAG |
| | SEQ ID NO: 137 | ACTA2 qPCR-R | AGGCATAGAGAGACAGCAC |
| CACNA2D1 | SEQ ID NO: 138 | CACNA2D1 qPCR-F | CTGGCCTAGCTCGATATTATCC |
| | SEQ ID NO: 139 | CACNA2D1 qPCR-R | ACACTTCCACTCACATCCAC |
| FAS | SEQ ID NO: 140 | FAS qPCR-F | GTATGTGAACACTGTGACCC |
| | SEQ ID NO: 141 | FAS qPCR-R | GCAAAAGAAGAAGACAAAGCC |
| PDGFRB | SEQ ID NO: 142 | PDGFRB qPCR-F | AGCAGTTCTACAATGCCATC |
| | SEQ ID NO: 143 | PDGFRB qPCR-R | TAACCTTCGCCCAACAGTC |
| SH3PXD2A | SEQ ID NO: 144 | SH3PXD2A qPCR-F | TTCCAACTGCCAAAGCCAC |
| | SEQ ID NO: 145 | SH3PXD2A qPCR-R | ATGACCTCTGCCTTCTGTCC |
| TAGLN | SEQ ID NO: 146 | TAGLN qPCR-F | CATGTTCCAGACTGTTGACC |
| | SEQ ID NO: 147 | TAGLN qPCR-R | CCTGCGCTTTCTTCATAAACC |
| ACP2 | SEQ ID NO: 148 | ACP2 qPCR-F | AACCTGACCCTAATGGCGAC |
| | SEQ ID NO: 149 | ACP2 qPCR-R | ATATGTGGCAGGAGGCGTAG |
| CTSD | SEQ ID NO: 150 | CTSD qPCR-F | GCAAACTGCTGGACATCGCTTG |
| | SEQ ID NO: 151 | CTSD qPCR-R | GCCATAGTGGATGTCAAACGAGG |
| MMP11 | SEQ ID NO: 152 | MMP11 qPCR-F | CGACTATGATGAGACCTGGAC |
| | SEQ ID NO: 153 | MMP11 qPCR-R | GTAGCGAAAGGTGTAGAAG |
| TPP1 | SEQ ID NO: 154 | TPP1 qPCR-F | GGTGGCTTCAGCAATGTGTTCC |
| | SEQ ID NO: 155 | TPP1 qPCR-R | GAAGTAACTGGATGGTGGCAGG |
| CD44 | SEQ ID NO: 156 | CD44 qPCR-F | AGCAACTGAGACAGCAACCA |
| | SEQ ID NO: 157 | CD44 qPCR-R | AGACGTACCAGCCATTTGTGT |
| ESM1 | SEQ ID NO: 158 | ESM1 qPCR-F | TTCAGTAACCAAGTCTTCCAAC |
| | SEQ ID NO: 159 | ESM1 qPCR-R | CAGCATTCTCTTTCACAACTTC |
| FOXD1 | SEQ ID NO: 160 | FOXD1 qPCR-F | CTCGTATATCGCGCTCATCA |
| | SEQ ID NO: 161 | FOXD1 qPCR-R | TCTTGACGAAGCAGTCGTTG |
| HEG1 | SEQ ID NO: 162 | HEG1 qPCR-F | CCCAAAAATCCTCGCTCAC |
| | SEQ ID NO: 163 | HEG1 qPCR-R | AGTCCGTTTCGTTCAAGTTC |
| TCF4 | SEQ ID NO: 164 | TCF4 qPCR-F | CAATCTCTCTCCACCTTTTGTC |
| | SEQ ID NO: 165 | TCF4 qPCR-R | TGCCCATATCCATGTCACC |
| VEGFC | SEQ ID NO: 166 | VEGFC qPCR-F | TCCACCACCAAACATGCAG |
| | SEQ ID NO: 167 | VEGFC qPCR-R | AAGGGACACAACGACACAC |
| ABL1 | SEQ ID NO: 168 | ABL1 qPCR-F | CCCTCCTTTGCTGAAATCC |
| | SEQ ID NO: 169 | ABL1 qPCR-R | CTCCTTTTCCACTTCGTCTG |
| APOLD1 | SEQ ID NO: 170 | APOLD1 qPCR-F | CAGATGCGAGAGATCCTGAG |
| | SEQ ID NO: 171 | APOLD1 qPCR-R | GCCAAAGAAGACGATGAAGTAG |
| BDNF | SEQ ID NO: 172 | BDNF qPCR-F | CGTGATAGAAGAGCTGTTGG |
| | SEQ ID NO: 173 | BDNF qPCR-R | CTTGACTACTGAGCATCACC |
| DZIP1 | SEQ ID NO: 174 | DZIP1 qPCR-F | GTCTTCAACTATTACGACCCC |
| | SEQ ID NO: 175 | DZIP1 qPCR-R | ACTTTTCACTGTGTTCTTCCC |
| LEF1 | SEQ ID NO: 176 | LEF1 qPCR-F | ACCAGATTCTTGGCAGAAGG |
| | SEQ ID NO: 177 | LEF1 qPCR-R | TGCAGACCAGCCTGGATAAA |
| MYC | SEQ ID NO: 178 | MYC qPCR-F | CCTGGTGCTCCATGAGGAGAC |
| | SEQ ID NO: 179 | MYC qPCR-R | CAGACTCTGACCTTTTGCCAGG |
| NAV1 | SEQ ID NO: 180 | NAV1 qPCR-F | TCAATGCCAACAAGGAAGAG |
| | SEQ ID NO: 181 | NAV1 qPCR-R | ACACGACAGAAAGAAGCAAG |
| NRP2 | SEQ ID NO: 182 | NRP2 qPCR-F | GATGATGAATACGAGGTGGAC |
| | SEQ ID NO: 183 | NRP2 qPCR-R | TGATGGTGATGAGGATGGG |
| PKDCC | SEQ ID NO: 184 | PKDCC qPCR-F | AAGCAGCAGTACCGAGTACC |
| | SEQ ID NO: 185 | PKDCC qPCR-R | TCTCACAGACATCCACAGCC |
| TMCC3 | SEQ ID NO: 186 | TMCC3 qPCR-F | CCGTGAATGCTAAAGTTCTCC |
| | SEQ ID NO: 187 | TMCC3 qPCR-R | GACACACACACTAAGATGACAG |
| CDA | SEQ ID NO: 188 | CDA qPCR-F | CTGAACGGACCGCTATCCAG |
| | SEQ ID NO: 189 | CDA qPCR-R | AGGCCCCACATGGAGAGATAA |

### <1-9> Time-Course Analysis, Quantification of Relative Motility of 5637GRC and CrM Signature

In 5637GRCA and 5637GRCB, the top 4,000 differentially expressed genes were identified based on standard deviation, and 2,673 genes commonly included were selected. For time-course analysis, two different datasets were combined using normalization, and differences between 5637GRCA and 5637GRCB were identified using principal component analysis (PCA) and an appropriate number of clusters was determined. Subsequently, data values from infiltration and migration experiments were used to quantify the relative motility of the 5637GRC cell line, and gene weight values were determined based on the relative motility of the 5637GRC cell line. The chemoresistance-motility signature (CrM signature) was calculated as a difference (−) between an average of a multiple of z-scores and weighted values of 52 genes included in a motility signature (M) stage and a chemoresistance and proliferation (C) stage and an average of a multiple of z-scores and weighted values of 11 genes included in a parent (P) stage.

### <1-10> Analysis of Major Biological Characteristics and Statistical Analysis

Significance criterion (p<0.05 or false discovery rate<0.25). The p-value estimated by Fisher's exact test determined whether there was a statistically significant overlap between genes in a dataset and genes regulated by a pathway. All analyses were performed three times and represented as mean±standard deviation (S.D). Clustering analysis was performed using a central correlation coefficient and a central linkage method using ComplexHeatmap (ver. 2.4.3) tools. A t-test was performed on two samples to determine the significance of a difference between subgroups. In addition, a Kaplan-Meier method was used to calculate cancer-specific survival or progression-free survival, and a log-rank test was used to evaluate a survival difference between the two groups. A Fisher's exact test was performed to compare categorical variables, and all statistical analyses were performed in the R language environment (ver. 3.6.3).

### <Example 2> Construction and Selection of Gemcitabine-Resistant Cancer Cell Lines

In the present disclosure, a 5637GRC cell line was constructed stepwise to characterize the molecular evolution resulting from the treatment with chemotherapy. Five candidate groups of the 5637 cell line were treated with 5 μM of GEM for 3 days and then allowed a recovery period. This process was referred to as stage 1 (P1), and the process was repeated up to phase 15 (P15) (A of FIG. 1). A process of configuring a cell line in each stage was shown in B of FIG. 1. In an early stage, a small number of cells survived after 3 days of GEM treatment, and the residual cells proliferated in the form of colonies during the recovery period. In a late stage, the proportion of surviving cells increased after GEM treatment and no more colonies were formed, but it was confirmed that the cells recovered at a faster rate than the cells in the early stage (B of FIG. 1). Resistance to GEM was evaluated in a P15 cell line of five 5637GRC candidate groups. A 5637 parental cell line (P0) was sensitive to GEM, but GRCA and GRCB cell lines were significantly highly resistant to GEM compared to other candidate groups (C of FIG. 1). Thereafter, the resistance to GEM was compared in cell lines at stages P0, P2, P3, P5, P7, P10, and P15 of GRCA and GRCB. It was confirmed that the resistance to GEM increased in a phase-dependent manner in both groups (D and E of FIG. 1). Subsequently, P0, P3, P7, and P15 cell lines from GRCA and GRCB groups were selected for RNA sequencing analysis.

### <Example 3> Confirmation of Increased Gemcitabine Resistance in Gemcitabine-Resistant Cancer Cell Lines

To confirm whether a 5637GRC cell line stably acquired stage-specific gemcitabine resistance, an MTT assay was performed. Specifically, in GRCA and GRCB groups, IC₅₀ values for GEM of P0, P3, P7, and P15 cell lines were evaluated. The IC₅₀ values of the 5637GRC cell line increased in a phase-dependent manner in both groups (A and B of FIG. 2). Colony formation capacity was evaluated by performing a clonogenic assay after GEM treatment, and the relative number of colonies increased in a phase-dependent manner in the GRCA group. Colony formation in the GRCB group was significantly increased in the P7 cell line and maintained until P15 (C of FIG. 2). In addition, as a result of analyzing the anchorage-independent growth capacity of surviving cells by soft agar assay after treatment with 1 μM GEM, the number of spheres formed increased in a phase-dependent manner in both 5637GRC groups (D of FIG. 2).

In addition, the proliferation characteristics of GRCA and GRCB groups without GEM treatment were evaluated by MTT assay. Cell viability in both 5637GRC groups increased in a phase-dependent manner (A and B of FIG. 3). The colony formation capacity of GRCA and GRCB cell lines also increased in a phase-dependent manner (C of FIG. 3). To further confirm in vivo tumorigenic capacity, cell lines in the GRCA group were injected subcutaneously into the flank of a 6-week-old BALB/c nude mouse. After 8 weeks, the tumor tissue formed in the flank was obtained and the size was measured. As a result, it was confirmed that the tumor volume significantly increased depending on a phase of the GRCA cell line (A to D of FIG. 4). In addition, when the obtained tumor tissue was embedded in paraffin, it was confirmed that the tumor tissue was composed of cancer cells, and a proliferation marker Ki67 in the tumor tissue was most highly expressed in the tumor formed from GRCA-P15 (E of FIG. 4). As the result, it was confirmed that GRCA and GRCB cell lines stably acquired GEM resistance and tumorigenicity.

### <Example 4> Confirmation of Cell Infiltration (Invasion) and Migration Capacity of Gemcitabine-Resistant Cancer Cell Lines

To evaluate the infiltration (invasion) and migration phenotypes in bladder cancer cell lines of the present disclosure, a Boyden chamber assay was performed. As a result, it was confirmed that the cell infiltration (invasion) and migration capacity of the GRCA cell lines increased in a phase-dependent manner from P0 to P7 and then decreased at P15 (A of FIG. 5). Meanwhile, the infiltration (invasion) and migration capacity of the GRCB cell lines significantly increased at P3 and then decreased at P7 and P15 (B of FIG. 5). In addition, a wound healing assay was performed to confirm the migration capacity of the GRCA and GRCB cell lines, and as a result, the wound closure of the GRCA cell lines increased in a phase-dependent manner from P0 to P7 and then decreased at P15 (C of FIG. 5). In the GRCB cell lines, the wound closure was highest at P3 compared to P0, and then decreased at P7 and P15 (D of FIG. 5).

A 3D microfluidic system includes cancer cells and vascular networks to confirm fundamental mechanisms associated with infiltration, angiogenesis, and metastasis. Therefore, the migration capacity of GRCA cell lines was further evaluated under 3D culture conditions such as an in vivo tumor microenvironment using a microfluidic device. Similarly to 2D culture conditions, as a result of measuring the area, average distance, and number of infiltrated cells by degrading a collagen substrate, it was confirmed that cell infiltration increased in P7 and decreased in P15 compared to P0 (A and B of FIG. 6).

In addition, it was confirmed whether the infiltration (invasion) and migration capacity of GRC cell lines increased in vivo metastasis. Specifically, the GRCA cell lines were intravenously injected into the tail of 6-week-old BALB/c nude mice. After 10 weeks, the mice were sacrificed and the tumor tissue of another site and lung tissue of the mice were isolated. As a result, in the same manner as an in vitro experiment, the mice injected with GRCA-P3 and P7 cells showed a significantly increased incidence of tumor metastasis in the lung and other sites (A and B of FIG. 7). In addition, it was confirmed that the expression of markers MMP2, MMP3, CAV1, and L1CAM associated with metastasis was highest in tumors derived from the P7 cell line metastasized to the lung (C of FIG. 7).

### <Example 5> Confirmation of Molecular Changes Due to Acquisition of Resistance in Gemcitabine-Resistant Cancer Cell Lines

To confirm the molecular changes during acquisition of GEM resistance of the present disclosure, RNA sequencing was performed on GRCA and GRCB cell lines. To perform time-course analysis of a molecular mechanism considering the characteristics of 5637GRC, three stages of 5637GRC were defined based on a cell phenotype: parental stage (P; P0), motility-signature (M; P3 and P7), and chemo-proliferation (C; P15). A flowchart of bioinformatics analysis was shown in A of FIG. 8. The abundant biological properties of common 2,673 genes (top 4,000 genes by standard deviation of each dataset) showing diverse expression across GRCA and GRCB were investigated, and functional analyses such as K-means clustering (K=8) and Gene Ontology (GO) and Kyoto encyclopedia of genes and genomes (KEGG) pathways were performed to identify relevant genes at each stage of the 5637GRC cell line. In stage P (G1 and G2), genes related to DNA replication and stress response (11 genes including CCND1, CDH12, ID3, CDKN1A, and MCM3) were up-regulated. At stage M (G3, G4, G5, and G6), focal adhesion, PI3K-AKT signaling pathway, cell adhesion, transcriptional regulation error in cancer, MAPK signaling pathway, pan-F-TBR, and lysosome-related genes (35 genes including CAV1, CAV2, L1CAM, LCN2, PDGFC, THBS3, ACTA2, CACNA2D1, SH3PXD2A, and TAGLN) were up-regulated. In stage C (G7 and G8), 5637GRC showed up-regulation of genes (16 genes including CD44, FOXD1, TCF4, BDNF, and MYC) associated with cell differentiation and MAPK signaling pathway (A of FIG. 9). In addition, CDA, which was directly related to the mechanism of action of gemcitabine, increased sequentially from stages P to C (A of FIG. 9).

Thereafter, based on the results of Example 4 above (A and B of FIG. 5), the motility of 5637GRC was quantified and the relative motility corresponding to four stages (0.98 for P0, 2.48 for P3, 2.41 for P7, and 1.50 for P15) was calculated. These values were determined as weighted values considering when 63 genes were activated. Genes in stage P were given a weighted value of 0.98, genes in G3 in stage M were given a weighted value of 2.45 (average of 2.48 and 2.41, Box 1), and genes in G4 to G6 in stage M were given a weighted value of 1.96 (average of 2.41 and 1.50, Box 2). Genes in stage C were given a weight of 1.50 (Box 3) (B of FIG. 9). A chemoresistance-motility (CrM) signature was used to estimate the prognostic relevance by applying the characteristics of 5637GRC to a clinical cohort. The CrM signature was calculated by subtracting an average of a multiple of z-scores and weighted values of 11 genes ATP8B1, CCND1, CDH12, FERMT1, HSPD1, HSPE1, ID3, MAML3, CDKN1A, ELF4 and MCM3 in stage P from an average of a multiple of z-scores and weighted values of 35 genes AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11 and TPP1 in stage M and 17 genes CD44, ESM1, FOXD1, HEG1, TCF4, VEGFC, ABL1, APOLD1, BDNF, DZIP1, LEF1, MYC, NAV1, NRP2, PKDCC, TMCC3 and CDA in stage C (B of FIG. 9). In addition, time-course analysis observed changes in several biological pathways at each stage. These results suggest that 5637GRC has activated various molecular mechanisms to evade gemcitabine treatment.

### <Example 6> Estimation of Prognostic Relevance of CrM Signature in Clinical Cohort of Bladder Cancer Patients

Since the 5637 cell line showed similar characteristics to NMIBC and was actually derived from low-grade bladder cancer cells, a gene expression pattern was confirmed based on the CrM signature in a UROMOL cohort consisting of NMIBC patients. The median value of the CrM signature divided the UROMOL cohort into two groups (CrM-high and CrM-low; A of FIG. 10). Significant differences in UROMOL classification, grade and stage ratios according to CrM signature were identified. Patients with high T1 grade were more included in a group with high CrM grade (p<0.001 by Chi-squared test, B of FIG. 10). Kaplan-Meier plots and log-rank tests indicated that subgroups significantly predicted progression-free survival (PFS) in NMIBC patients based on the CrM signature (p=0.001 by log-rank test, C of FIG. 10). In addition, survival analysis was conducted to confirm differences in prognosis by stage and grade. Interestingly, the prognostic relevance was observed in patients with lower stage and grade (p=0.007 by log-rank test, C of FIG. 10). These results suggested that NMIBC patients with high CrM signature and relatively low risk may undergo molecular evolution toward a potentially high-risk group.

A TCGA cohort consisting of MIBC patients was also classified into two groups based on a CrM signature (CrM-high and CrM-low; A of FIG. 11). The activation of several proteins E-cadherin, claudin7, GATA3, fibronectin, caveolin, PAI1, and YAP1 supports the observation of metastasis in the TCGA cohort (89 of 133 patients, 67%, p<0.001) (B of FIG. 11). To investigate the association between TCGA classification and CrM signature, a distribution of five molecular subtypes in each group was investigated (p<0.001 by Fisher's exact test, C of FIG. 11). A higher proportion of luminal infiltration and basal/squamous subtypes was observed in patients in a CrM-high group (45 of 56 patients, 78% and 70 of 78 patients, 89%) and a higher proportion of luminal papillary subtypes was observed in patients in a CrM-low group (96 of 100 patients, 96%) (C of FIG. 11). In particular, the patients in the high CrM group had a poor prognosis (p=0.001 in the log-rank test, D of FIG. 11).

The present disclosure confirmed that chemotherapy enhanced cell motility in NMIBC 5637 cell lines. As the result of the present disclosure, it was confirmed that 5637 GRC exhibited enhanced lysosomal, PI3K-AKT, TGF-β and MAPK signaling pathways during stages M and C to induce increased cell motility and evasion of gemcitabine treatment. In addition, the prognostic relevance was confirmed by establishing the changes in the stepwise molecular mechanism of 5637 GRC and applying the CrM signature to a clinical cohort. It was suggested that these findings may be useful in understanding the stepwise molecular mechanisms for acquiring chemotherapy resistance and improving prognosis in bladder cancer patients.

According to the present invention, it was confirmed that bladder cancer cell lines increase in infiltration (invasion) and migration capacity while acquiring anti-cancer drug resistance, and it was confirmed that the expression of ATP8B, CCND1, CDH12, FERMT1, HSPD1, HSPE1, ID3, MAML3, CDKNV1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSC5D, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA is increased in bladder cancer cells with increased infiltration (invasion) and metastasis. In addition, it was confirmed that selected genes are associated with metastasis and prognosis of cancer in bladder cancer patients.

### [National Research and Development Project Supporting the Invention]

[Project Unique Number] 1711143981
[Project Number] 2020R1A2C1007356
[Ministry name] Ministry of Science and ICT
[Project Management (Special) Institution Name] National Research Foundation of Korea
[Research Project Name] Mid-sized researcher support project
[Research Subject Name] Discovery of factors interacting with STC1 and DDR1 genes involved in transverse mutations in anticancer drug resistance and identification of their molecular mechanisms
[Percent Contribution] ½
[Project performance institute name] Dong-A University Industry-Academic Cooperation Group
[Research Period] Mar. 1, 2020 to Feb. 28, 2023
[National research and development project supporting the invention]
[Project Unique Number] 2710012464
[Project Number] 2023R1A2C1002940
[Ministry name] Ministry of Science and ICT
[Project Management (Special) Institution Name] National Research Foundation of Korea
[Research Project Name] Personal basic research (Ministry of Science and ICT)
[Research Subject Name] Characterization and application of companion diagnostic biomarkers through secretory protein analysis for stepwise acquisition of anticancer drug resistance
[Project performance institute name] Dong-A University
[Research Period] Mar. 1, 2024 to Feb. 28, 2025

1. A biomarker of metastatic cancer comprising a gene selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

2. The biomarker of claim 1, wherein the ATP8B1 includes a base sequence of SEQ ID NO: 1;
CCND1 includes a base sequence of SEQ ID NO: 2;
CDH12 includes a base sequence of SEQ ID NO: 3;
FERMT1 includes a base sequence of SEQ ID NO: 4;
HSPD1 includes a base sequence of SEQ ID NO: 5;
HSPE1 includes a base sequence of SEQ ID NO: 6;
ID3 includes a base sequence of SEQ ID NO: 7;
MAML3 includes a base sequence of SEQ ID NO: 8;
CDKN1A includes a base sequence of SEQ ID NO: 9;
ELF4 includes a base sequence of SEQ ID NO: 10;
MCM3 includes a base sequence of SEQ ID NO: 11;
AREG includes a base sequence of SEQ ID NO: 12;
BMP6 includes a base sequence of SEQ ID NO: 13;
CAV1 includes a base sequence of SEQ ID NO: 14;
CAV2 includes a base sequence of SEQ ID NO: 15;
COL8A2 includes a base sequence of SEQ ID NO: 16;
CTSK includes a base sequence of SEQ ID NO: 17;
EBI3 includes a base sequence of SEQ ID NO: 18;
EREG includes a base sequence of SEQ ID NO: 19;
GSN includes a base sequence of SEQ ID NO: 20;
ITGA2B includes a base sequence of SEQ ID NO: 21;
JAK3 includes a base sequence of SEQ ID NO: 22;
L1CAM includes a base sequence of SEQ ID NO: 23;
LAMA3 includes a base sequence of SEQ ID NO: 24;
LAMC2 includes a base sequence of SEQ ID NO: 25;
LCN2 includes a base sequence of SEQ ID NO: 26;
OLFML2A includes a base sequence of SEQ ID NO: 27;
PDGFC includes a base sequence of SEQ ID NO: 28;
PRKCG includes a base sequence of SEQ ID NO: 29;
SSCSD includes a base sequence of SEQ ID NO: 30;
THBS3 includes a base sequence of SEQ ID NO: 31;
VASN includes a base sequence of SEQ ID NO: 32;
MMP3 includes a base sequence of SEQ ID NO: 33;
SERPINB2 includes a base sequence of SEQ ID NO: 34;
VCAN includes a base sequence of SEQ ID NO: 35;
ZP3 includes a base sequence of SEQ ID NO: 36;
ACTA2 includes a base sequence of SEQ ID NO: 37;
CACNA2D1 includes a base sequence of SEQ ID NO: 38;
FAS includes a base sequence of SEQ ID NO: 39;
PDGFRB includes a base sequence of SEQ ID NO: 40;
SH3PXD2A includes a base sequence of SEQ ID NO: 41;
TAGLN includes a base sequence of SEQ ID NO: 42;
ACP2 includes a base sequence of SEQ ID NO: 43;
CTSD includes a base sequence of SEQ ID NO: 44;
MMP11 includes a base sequence of SEQ ID NO: 45;
TPP1 includes a base sequence of SEQ ID NO: 46;
CD44 includes a base sequence of SEQ ID NO: 47;
ESM1 includes a base sequence of SEQ ID NO: 48;
FOXD1 includes a base sequence of SEQ ID NO: 49;
HEG1 includes a base sequence of SEQ ID NO: 50;
TCF4 includes a base sequence of SEQ ID NO: 51;
VEGFC includes a base sequence of SEQ ID NO: 52;
ABL1 includes a base sequence of SEQ ID NO: 53;
APOLD1 includes a base sequence of SEQ ID NO: 54;
BDNF includes a base sequence of SEQ ID NO: 55;
DZIP1 includes a base sequence of SEQ ID NO: 56;
LEF1 includes a base sequence of SEQ ID NO: 57;
MYC includes a base sequence of SEQ ID NO: 58;
NAV1 includes a base sequence of SEQ ID NO: 59;
NRP2 includes a base sequence of SEQ ID NO: 60;
PKDCC includes a base sequence of SEQ ID NO: 61;
TMCC3 includes a base sequence of SEQ ID NO: 62; and
CDA includes a base sequence of SEQ ID NO: 63.

3. (canceled)

4. The biomarker of claim 1, wherein when the expression of the gene is increased compared to a reference value of a control group, metastasis of cancer cells increases.

5. The biomarker of claim 1, wherein when the expression of the gene is increased compared to the reference value of the control group, a poor prognosis is exhibited.

6. The biomarker of claim 5, wherein the poor prognosis is reduced metastasis of cancer or viability of a subject.

7. The biomarker of claim 1, wherein the cancer is selected from the group consisting of bladder cancer, stomach cancer, colon cancer, rectal cancer, anal cancer, bone cancer, cerebrospinal tumor, head and neck cancer, thymoma, mesothelioma, esophageal cancer, biliary tract cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer.

8. A biomarker composition for predicting metastasis or prognosis of cancer comprising a preparation for measuring the expression level of the gene according to claim 1 selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

9. The biomarker composition of claim 8, wherein the preparation for measuring the expression level of the gene includes a primer pair selected from the group consisting of a primer pair of SEQ ID NOs: 64 and 65; a primer pair of SEQ ID NOs: 66 and 67; a primer pair of SEQ ID NOs: 68 and 69; a primer pair of SEQ ID NOs: 70 and 71; a primer pair of SEQ ID NOs: 72 and 73; a primer pair of SEQ ID NOs: 74 and 75; a primer pair of SEQ ID NOs: 76 and 77; a primer pair of SEQ ID NOs: 78 and 79; a primer pair of SEQ ID NOs: 80 and 81; a primer pair of SEQ ID NOs: 82 and 83; a primer pair of SEQ ID NOs: 84 and 85; a primer pair of SEQ ID NOs: 86 and 87; a primer pair of SEQ ID NOs: 88 and 89; a primer pair of SEQ ID NOs: 90 and 91; a primer pair of SEQ ID NOs: 92 and 93; a primer pair of SEQ ID NOs: 94 and 95; a primer pair of SEQ ID NOs: 96 and 97; a primer pair of SEQ ID NOs: 98 and 99; a primer pair of SEQ ID NOs: 100 and 101; a primer pair of SEQ ID NOs: 102 and 103; a primer pair of SEQ ID NOs: 104 and 105; a primer pair of SEQ ID NOs: 106 and 107; a primer pair of SEQ ID NOs: 108 and 109; a primer pair of SEQ ID NOs: 110 and 111; a primer pair of SEQ ID NOs: 112 and 113; a primer pair of SEQ ID NOs: 114 and 115; a primer pair of SEQ ID NOs: 116 and 117; a primer pair of SEQ ID NOs: 118 and 119; a primer pair of SEQ ID NOs: 120 and 121; a primer pair of SEQ ID NOs: 122 and 123; a primer pair of SEQ ID NOs: 124 and 125; a primer pair of SEQ ID NOs: 126 and 127; a primer pair of SEQ ID NOs: 128 and 129; a primer pair of SEQ ID NOs: 130 and 131; a primer pair of SEQ ID NOs: 132 and 133; a primer pair of SEQ ID NOs: 134 and 135; a primer pair of SEQ ID NOs: 136 and 137; a primer pair of SEQ ID NOs: 138 and 139; a primer pair of SEQ ID NOs: 140 and 141; a primer pair of SEQ ID NOs: 142 and 143; a primer pair of SEQ ID NOs: 144 and 145; a primer pair of SEQ ID NOs: 146 and 147; a primer pair of SEQ ID NOs: 148 and 149; a primer pair of SEQ ID NOs: 150 and 151; a primer pair of SEQ ID NOs: 152 and 153; a primer pair of SEQ ID NOs: 154 and 155; a primer pair of SEQ ID NOs: 156 and 157; a primer pair of SEQ ID NOs: 158 and 159; a primer pair of SEQ ID NOs: 160 and 161; a primer pair of SEQ ID NOs: 162 and 163; a primer pair of SEQ ID NOs: 164 and 165; a primer pair of SEQ ID NOs: 166 and 167; a primer pair of SEQ ID NOs: 168 and 169; a primer pair of SEQ ID NOs: 170 and 171; a primer pair of SEQ ID NOs: 172 and 173; a primer pair of SEQ ID NOs: 174 and 175; a primer pair of SEQ ID NOs: 176 and 177; a primer pair of SEQ ID NOs: 178 and 179; a primer pair of SEQ ID NOs: 180 and 181; a primer pair of SEQ ID NOs: 182 and 183; a primer pair of SEQ ID NOs: 184 and 185; a primer pair of SEQ ID NOs: 186 and 187; and a primer pair of SEQ ID NOs: 188 and 189.

10. The biomarker composition of claim 8, wherein the measuring of the expression level of the gene is measuring the amount of mRNA transcribed by the gene or the amount of protein encoded by the gene.

11. A kit for predicting metastasis or prognosis of cancer comprising the composition of claim 8.

12. A method of providing information for predicting metastasis or prognosis of cancer comprising: confirming the expression level of the gene according to claim 1 selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA;
comparing the expression level of the gene with a reference value of a control group; and
determining that the cancer metastasis has increased when the expression level increases compared to the reference value of the control group.

13. The method of claim 12, wherein the biological sample is at least one selected from the group consisting of blood, hair, saliva, epidermis, semen, vaginal exudate, isolated cells, tissue samples, dandruff, and bones.

14. The method of claim 12, wherein the expression level of the gene is measured by a method selected from the group consisting of reverse transcriptase-polymerase chain reaction, real time-polymerase chain reaction, Western blot, Northern blot, enzyme linked immunosorbent assay (ELISA), radioimmunoassay (RIA), radioimmunodiffusion, and immunoprecipitation assay.

15. A screening method of a metastatic cancer therapeutic agent comprising:
treating cells with a candidate material;
confirming the expression level of the gene according to claim 1 selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA in the cells treated with the candidate material;
comparing the expression level of the gene with a reference value of a control group; and
selecting a therapeutic agent for metastatic cancer when the expression level of the gene is reduced compared to the reference value of the control group.

16. The method of claim 15, wherein the cells are cancer cells.

17. The method of claim 16, wherein the cancer is selected from the group consisting of bladder cancer, stomach cancer, colon cancer, rectal cancer, anal cancer, bone cancer, cerebrospinal tumor, head and neck cancer, thymoma, mesothelioma, esophageal cancer, biliary tract cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer.

18. A biomarker of cancer having drug resistance to gemcitabine comprising the gene according to claim 1 selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.

19. (canceled)

20. The biomarker of claim 18, wherein when the expression of the gene is increased compared to a reference value of a control group, metastasis of cancer cells increases.

21. The method of claim 18, wherein when the expression of the gene is increased compared to the reference value of the control group, a poor prognosis is exhibited.

22. The method of claim 21, wherein the poor prognosis is reduced metastasis of cancer or viability of a subject.

23. The method of claim 18, wherein the cancer is selected from the group consisting of bladder cancer, stomach cancer, colon cancer, rectal cancer, anal cancer, bone cancer, cerebrospinal tumor, head and neck cancer, thymoma, mesothelioma, esophageal cancer, biliary tract cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer.

24. A biomarker composition for diagnosis of cancer having drug resistance to gemcitabine, comprising a preparation for measuring the expression level of the gene according to claim 1 selected from the group consisting of ATP8B1, CCND1, CDH12, FERMT1, HSPD1, HSPE1, ID3, MAML3, CDKN1A, ELF4, MCM3, AREG, BMP6, CAV1, CAV2, COL8A2, CTSK, EBI3, EREG, GSN, ITGA2B, JAK3, L1CAM, LAMA3, LAMC2, LCN2, OLFML2A, PDGFC, PRKCG, SSCSD, THBS3, VASN, MMP3, SERPINB2, VCAN, ZP3, ACTA2, CACNA2D1, FAS, PDGFRB, SH3PXD2A, TAGLN, ACP2, CTSD, MMP11, TPP1, CD44, ESM1, FOXD1, HEG1, TCF4, VEGFC, ABL1, APOLD1, BDNF, DZIP1, LEF1, MYC, NAV1, NRP2, PKDCC, TMCC3, and CDA.


SEQUENCE LISTING (189 sequences)

SEQ ID NO: 1 - DNA, 152 bp, synthetic construct
  source 1..152: mol_type = other DNA
cctctcaacacaaccacaacaaaatagagcaagttgattttagctggaatacatatgctgatgggaagcttgcattttatgaccactatcttattgagcaaatccagtcagggaaagagccagaagtacgacagttcttcttcttgctcgca

SEQ ID NO: 2 - DNA, 92 bp, synthetic construct
  source 1..92: mol_type = other DNA
aggcggaggagaacaaacagatcatccgcaaacacgcgcagaccttcgttgccctctgtgccacagatgtgaagttcatttccaatccgccc

SEQ ID NO: 3 - DNA, 115 bp, synthetic construct
  source 1..115: mol_type = other DNA
cctgaggctgctatcaaaccaaattttacagttcgtgacttcagaaacaacacagcggggattgaaacccgaagaaatggatacagccgcaggcagcaagagttgtatttcctcc

SEQ ID NO: 4 - DNA, 159 bp, synthetic construct
  source 1..159: mol_type = other DNA
accacggtgtgcaaaaagacacaaatccaaacagctggccgcccggatcctggaggcgcaccagaacgtggcccagatgcccctggtcgaagccaagctgcggttcatccaggcgtggcagtcactgcctgagtttggcctcacctactaccttgtcag

SEQ ID NO: 5 - DNA, 146 bp, synthetic construct
  source 1..146: mol_type = other DNA
gaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgg

SEQ ID NO: 6 - DNA, 126 bp, synthetic construct
  source 1..126: mol_type = other DNA
gagagattcaaccagttagcgtgaaagttggagataaagttcttctcccagaatatggaggcaccaaagtagttctagatgacaaggattatttcctatttagagatggtgacattcttggaaagt

SEQ ID NO: 7 - DNA, 122 bp, synthetic construct
  source 1..122: mol_type = other DNA
tcatcgactacattctcgacctgcaggtagtcctggccgagccagcccctggaccccctgatggcccccaccttcccatccagacagccgagctcactccggaacttgtcatctccaacgac

SEQ ID NO: 8 - DNA, 177 bp, synthetic construct
  source 1..177: mol_type = other DNA
tgcagcaatcacatctaccccggcagcacctccagccacagcggaatccatacccagtgcagcaggtcaatcagtttcaaggttctccccaggatatagcagccgtaagaagccaagcagccctccagagcatgcgaacgtcacggctgatggcacagaacgcaggcatgatgggaa

SEQ ID NO: 9 - DNA, 159 bp, synthetic construct
  source 1..159: mol_type = other DNA
aacaaagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatgg

SEQ ID NO: 10 - DNA, 123 bp, synthetic construct
  source 1..123: mol_type = other DNA
aagaccaagggcaaccgaagtacctcacctgtcactgaccccagcatccccattaggaagaaatcaaaggatggcaaaggcagcaccatctatctgtgggagttcctcctggctcttctgcaa

SEQ ID NO: 11 - DNA, 103 bp, synthetic construct
  source 1..103: mol_type = other DNA
acactccaaagacggcagactcacaggagaccaaggaatcccagaaagtggagttgagtgaatccaggttgaaggcattcaaggtggccctcttggatgtgtt

SEQ ID NO: 12 - DNA, 110 bp, synthetic construct
  source 1..110: mol_type = other DNA
ctggattggacctcaatgacacctactctgggaagcgtgaaccattttctggggaccacagtgctgatggatttgaggttacctcaagaagtgagatgtcttcagggagt

SEQ ID NO: 13 - DNA, 164 bp, synthetic construct
  source 1..164: mol_type = other DNA
gatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaa

SEQ ID NO: 14 - DNA, 112 bp, synthetic construct
  source 1..112: mol_type = other DNA
tcaaccgcgaccctaaacacctcaacgatgacgtggtcaagattgactttgaagatgtgattgcagaaccagaagggacacacagttttgacggcatttggaaggccagctt

SEQ ID NO: 15 - DNA, 110 bp, synthetic construct
  source 1..110: mol_type = other DNA
gaattctctttgccaccctcagctgtctgcacatctggattttaatgccttttgtaaagacctgcctaatggttctgccttcagtgcagacaatatggaagagtgtgaca

SEQ ID NO: 16 - DNA, 139 bp, synthetic construct
  source 1..139: mol_type = other DNA
acaagaagggctacctggaccaggcatctggtggggccgtgctccagctgcggcccaacgaccaggtctgggtgcagatgccgtcggaccaggccaacggcctctactccacggagtacatccactcctccttttcagg

SEQ ID NO: 17 - DNA, 139 bp, synthetic construct
  source 1..139: mol_type = other DNA
ttcccgcagtaatgacaccctttatatcccagaatgggaaggtagagccccagactctgtcgactatcgaaagaaaggatatgttactcctgtcaaaaatcagggtcagtgtggttcctgttgggcttttagctctgtg

SEQ ID NO: 18 - DNA, 157 bp, synthetic construct
  source 1..157: mol_type = other DNA
gatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagt

SEQ ID NO: 19 - DNA, 150 bp, synthetic construct
  source 1..150: mol_type = other DNA
ggacatgagtcaaaactactgcaggtgtgaagtgggttatactggtgtccgatgtgaacacttctttttaaccgtccaccaacctttaagcaaagaatatgtggctttgaccgtgattcttattattttgtttcttatcacagtcgtcgg

SEQ ID NO: 20 - DNA, 132 bp, synthetic construct
  source 1..132: mol_type = other DNA
agctggatgactacctgaacggccgggccgtgcagcaccgtgaggtccagggcttcgagtcggccaccttcctaggctacttcaagtctggcctgaagtacaagaaaggaggtgtggcatcaggattcaagc

SEQ ID NO: 21 - DNA, 141 bp, synthetic construct
  source 1..141: mol_type = other DNA
tgacaacggatacccagacctgatcgtgggagcttacggggccaaccaggtggctgtgtacagagctcagccagtggtgaaggcctctgtccagctactggtgcaagattcactgaatcctgctgtgaagagctgtgtcct

SEQ ID NO: 22 - DNA, 174 bp, synthetic construct
  source 1..174: mol_type = other DNA
tcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcat

SEQ ID NO: 23 - DNA, 157 bp, synthetic construct
  source 1..157: mol_type = other DNA
atgcctacatctacgttgtccagctgccagccaagatcctgactgcggacaatcagacgtacatggctgtccagggcagcactgcctaccttctgtgcaaggccttcggagcgcctgtgcccagtgttcagtggctggacgaggatgggacaacagt

SEQ ID NO: 24 - DNA, 118 bp, synthetic construct
  source 1..118: mol_type = other DNA
cttaccacctactgaccacctccaggcctcatttggatttcagacctttcaacccagtggcatattattagatcatcagacatggacaaggaacctgcaggtcactctggaagatggt

SEQ ID NO: 25 - DNA, 117 bp, synthetic construct
  source 1..117: mol_type = other DNA
tcactctcaagcctggtaaccaggcatatggatgagttcaagcgtacacagaagaatctgggaaactggaaagaagaagcacagcagctcttacagaatggaaaaagtgggagagag

SEQ ID NO: 26 - DNA, 83 bp, synthetic construct
  source 1..83: mol_type = other DNA
agacaaagacccgcaaaagatgtatgccaccatctatgagctgaaagaagacaagagctacaatgtcacctccgtcctgttta

SEQ ID NO: 27 - DNA, 101 bp, synthetic construct
  source 1..101: mol_type = other DNA
gacatcagcaagtatggcagtgtgcagaaaagctttgcagacagaggcctcccaaaacctcccaaggagaagctgcttcaggtggagaagctgagaaagga

SEQ ID NO: 28 - DNA, 96 bp, synthetic construct
  source 1..96: mol_type = other DNA
gcacacctcgtaacttctcagtgtccataagggaagaactaaagagaaccgataccattttctggccaggttgtctcctggttaaacgctgtggtg

SEQ ID NO: 29 - DNA, 89 bp, synthetic construct
  source 1..89: mol_type = other DNA
cgaagtcaagagccacaagttcaccgctcgcttcttcaagcagcccaccttctgcagccactgcaccgacttcatctggggtatcggaa

SEQ ID NO: 30 - DNA, 139 bp, synthetic construct
  source 1..139: mol_type = other DNA
actccatctcagaccccttcagctggagctggattcctggactggggagagatcgggatgcctggctcccgggagagctggccaccaagccctctgcaagtgtgactgccagtgttctggagaaaacaaccacgaaggc

SEQ ID NO: 31 - DNA, 123 bp, synthetic construct
  source 1..123: mol_type = other DNA
ccaacccactacagacagacagggatgaggacggggtgggagatgcttgcgacagctgccctgaaatgagcaatcctacccagacagatgcagacagcgacctggtgggggatgtctgtgata

SEQ ID NO: 32 - DNA, 131 bp, synthetic construct
  source 1..131: mol_type = other DNA
tctcacctatcgcaacctatcgggccctgataagcggctggtgacgctgcgactgcctgcctcgctcgctgagtacacggtcacccagctgcggcccaacgccacttactccgtctgtgtcatgcctttgg

SEQ ID NO: 33 - DNA, 151 bp, synthetic construct
  source 1..151: mol_type = other DNA
aggctttcccaagcaaatagctgaagactttccagggattgactcaaagattgatgctgtttttgaagaatttgggttcttttatttctttactggatcttcacagttggagtttgacccaaatgcaaagaaagtgacacacactttgaag

SEQ ID NO: 34 - DNA, 135 bp, synthetic construct
  source 1..135: mol_type = other DNA
attactcctcagaaccccaggcagtagacttcctagaatgtgcagaagaagctagaaaaaagattaattcctgggtcaagactcaaaccaaaggcaaaatcccaaacttgttacctgaaggttctgtagatgggg

SEQ ID NO: 35 - DNA, 140 bp, synthetic construct
  source 1..140: mol_type = other DNA
gaatgtcactctaatccctgtcgtaatggagccacttgtgttgatggttttaacacattcaggtgcctctgccttccaagttatgttggtgcactttgtgagcaagataccgagacatgtgactatggctggcacaaatt

SEQ ID NO: 36 - DNA, 162 bp, synthetic construct
  source 1..162: mol_type = other DNA
ttccactttgctaatgactccagaaacatgatatacatcacctgccacctgaaggtcaccctagctgagcaggacccagatgaactcaacaaggcctgttccttcagcaagccttccaacagctggttcccagtggaaggctcggctgacatctgtcaatgc

SEQ ID NO: 37 - DNA, 299 bp, synthetic construct
  source 1..299: mol_type = other DNA
gggaatgggacaaaaagacagctacgtgggtgacgaagcacagagcaaaagaggaatcctgaccctgaagtacccgatagaacatggcatcatcaccaactgggacgacatggaaaagatctggcaccactctttctacaatgagcttcgtgttgcccctgaagagcatcccaccctgctcacggaggcacccctgaaccccaaggccaaccgggagaaaatgactcaaattatgtttgagactttcaatgtcccagccatgtatgtggctatccaggcggtgctgtctctctatgcct

SEQ ID NO: 38 - DNA, 157 bp, synthetic construct
  source 1..157: mol_type = other DNA
ctggcctagctcgatattatccagcttcaccatgggttgataatagtagaactccaaataagattgacctttatgatgtacgcagaagaccatggtacatccaaggagctgcatctcctaaagacatgcttattctggtggatgtgagtggaagtgt

SEQ ID NO: 39 - DNA, 133 bp, synthetic construct
  source 1..133: mol_type = other DNA
gtatgtgaacactgtgacccttgcaccaaatgtgaacatggaatcatcaaggaatgcacactcaccagcaacaccaagtgcaaagaggaaggatccagatctaacttggggtggctttgtcttcttcttttgc

SEQ ID NO: 40 - DNA, 166 bp, synthetic construct
  source 1..166: mol_type = other DNA
agcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggtta

SEQ ID NO: 41 - DNA, 122 bp, synthetic construct
  source 1..122: mol_type = other DNA
ttccaactgccaaagccaccagagcccccttctgttgaggtggagtactacaccattgccgaattccagtcgtgcatttccgatggcatcagctttcggggtggacagaaggcagaggtcat

SEQ ID NO: 42 - DNA, 143 bp, synthetic construct
  source 1..143: mol_type = other DNA
catgttccagactgttgacctctttgaaggcaaagacatggcagcagtgcagaggaccctgatggctttgggcagcttggcagtgaccaagaatgatgggcactaccgtggagatcccaactggtttatgaagaaagcgcagg

SEQ ID NO: 43 - DNA, 139 bp, synthetic construct
  source 1..139: mol_type = other DNA
aacctgaccctaatggcgaccacctcccagctccccaagctgctggtttactctgcgcacgacactaccctggttgccctgcaaatggcactggatgtctacaatggtgaacaagccccctacgcctcctgccacatat

SEQ ID NO: 44 - DNA, 101 bp, synthetic construct
  source 1..101: mol_type = other DNA
gcaaactgctggacatcgcttgctggatccaccacaagtacaacagcgacaagtccagcacctacgtgaagaatggtacctcgtttgacatccactatggc

SEQ ID NO: 45 - DNA, 145 bp, synthetic construct
  source 1..145: mol_type = other DNA
cgactatgatgagacctggactatcggggatgaccagggcacagacctgctgcaggtggcagcccatgaatttggccacgtgctggggctgcagcacacaacagcagccaaggccctgatgtccgccttctacacctttcgctac

SEQ ID NO: 46 - DNA, 99 bp, synthetic construct
  source 1..99: mol_type = other DNA
ggtggcttcagcaatgtgttcccacggccttcataccaggaggaagctgtaacgaagttcctgagctctagcccccacctgccaccatccagttacttc

SEQ ID NO: 47 - DNA, 115 bp, synthetic construct
  source 1..115: mol_type = other DNA
agcaactgagacagcaaccaagaggcaagaaacctgggattggttttcatggttgtttctaccatcagagtcaaagaatcatcttcacacaacaacacaaatggctggtacgtct

SEQ ID NO: 48 - DNA, 104 bp, synthetic construct
  source 1..104: mol_type = other DNA
ttcagtaaccaagtcttccaacagatttgtttctctcacggagcatgacatggcatctggagatggcaatattgtgagagaagaagttgtgaaagagaatgctg

SEQ ID NO: 49 - DNA, 173 bp, synthetic construct
  source 1..173: mol_type = other DNA
ctcgtatatcgcgctcatcactatggccatcctgcagagccccaagaagcggctgacgctgagcgagatctgtgagttcatcagcggccgcttcccctactaccgggagaagttccccgcctggcagaacagcatccgccacaacctctcgctcaacgactgcttcgtcaaga

SEQ ID NO: 50 - DNA, 143 bp, synthetic construct
  source 1..143: mol_type = other DNA
cccaaaaatcctcgctcacaagaatggggccgagaagctattgaaatgcatgagaatggaagtaccaaaaacctcctccagatgacggatgtgtactactcgcctacaagtgtaaggaatccagaacttgaacgaaacggact

SEQ ID NO: 51 - DNA, 137 bp, synthetic construct
  source 1..137: mol_type = other DNA
caatctctctccaccttttgtcaattccagaatacaaagtaaaacagaaaggggctcatactcatcttatgggagagaatcaaacttacagggttgccaccagcagagtctccttggaggtgacatggatatgggca

SEQ ID NO: 52 - DNA, 105 bp, synthetic construct
  source 1..105: mol_type = other DNA
tccaccaccaaacatgcagctgttacagacggccatgtacgaaccgccagaaggcttgtgagccaggattttcatatagtgaagaagtgtgtcgttgtgtccctt

SEQ ID NO: 53 - DNA, 78 bp, synthetic construct
  source 1..78: mol_type = other DNA
ccctcctttgctgaaatccaccaagcctttgaaacaatgttccaggaatccagtatctcagacgaagtggaaaaggag

SEQ ID NO: 54 - DNA, 135 bp, synthetic construct
  source 1..135: mol_type = other DNA
cagatgcgagagatcctgagctgcctcgagtttttctgccgctggcagggctgcggggaccgccagctgctgcagtgcgggaggaacgcctccatcgccctgtacaattctgtctacttcatcgtcttctttggc

SEQ ID NO: 55 - DNA, 104 bp, synthetic construct
  source 1..104: mol_type = other DNA
cgtgatagaagagctgttggatgaggaccagaaagttcggcccaatgaagaaaacaataaggacgcagacttgtacacgtccagggtgatgctcagtagtcaag

SEQ ID NO: 56 - DNA, 145 bp, synthetic construct
  source 1..145: mol_type = other DNA
gtcttcaactattacgacccctccttttagttcagaggaggagcaggaggacgacgacctcatccgggcatacgcatccccaggcccacttcctgtgccgccaccacaaaacaagggcagcttcgggaagaacacagtgaaaagt

SEQ ID NO: 57 - DNA, 116 bp, synthetic construct
  source 1..116: mol_type = other DNA
accagattcttggcagaaggtggcatgccctctcccgtgaagagcaggctaaatattatgaattagcacggaaagaaagacagctacatatgcagctttatccaggctggtctgca

SEQ ID NO: 58 - DNA, 128 bp, synthetic construct
  source 1..128: mol_type = other DNA
cctggtgctccatgaggagacaccgcccaccaccagcagcgactctgaggaggaacaagaagatgaggaagaaatcgatgttgtttctgtggaaaagaggcaggctcctggcaaaaggtcagagtctg

SEQ ID NO: 59 - DNA, 131 bp, synthetic construct
  source 1..131: mol_type = other DNA
tcaatgccaacaaggaagagctgcttcgggtgctcgactgggtacccaagctgtggtatcatctccacaccttccttgagaagcacagcacctcagacttcctcatcggcccttgcttctttctgtcgtgt

SEQ ID NO: 60 - DNA, 121 bp, synthetic construct
  source 1..121: mol_type = other DNA
gatgatgaatacgaggtggactggagcaattcttcttctgcaacctcagggtctggcgccccctcgaccgacaaagaaaagagctggctgtacaccctggatcccatcctcatcaccatca

SEQ ID NO: 61 - DNA, 137 bp, synthetic construct
  source 1..137: mol_type = other DNA
aagcagcagtaccgagtaccagtgtatcccagacagcaccatcccccaggaagactaccgctgctggccatcctaccaccacgggagctgcctcctttcagtgttcaacctggctgaggctgtggatgtctgtgaga

SEQ ID NO: 62 - DNA, 79 bp, synthetic construct
  source 1..79: mol_type = other DNA
ccgtgaatgctaaagttctcctggggaggtgcatcaacgtgatcctggccttcatgactgtcatcttagtgtgtgtgtc

SEQ ID NO: 63 - DNA, 108 bp, synthetic construct
  source 1..108: mol_type = other DNA
ctgaacggaccgctatccagaaggccgtctcagaagggtacaaggatttcagggcaattgctatcgccagtgacatgcaagatgattttatctctccatgtggggcct

SEQ ID NO: 64 - DNA, 20 bp, synthetic construct
  source 1..20: mol_type = other DNA
cctctcaacacaaccacaac

SEQ ID NO: 65 - DNA, 19 bp, synthetic construct
  source 1..19: mol_type = other DNA
tgcgagcaagaagaagaac

SEQ ID NO: 66 - DNA, 18 bp, synthetic construct
  source 1..18: mol_type = other DNA
aggcggaggagaacaaac

SEQ ID NO: 67 - DNA, 19 bp, synthetic construct
  source 1..19: mol_type = other DNA
gggcggattggaaatgaac

SEQ ID NO: 68 - DNA, 19 bp, synthetic construct
  source 1..19: mol_type = other DNA
cctgaggctgctatcaaac

SEQ ID NO: 69 - DNA, 22 bp, synthetic construct
  source 1..22: mol_type = other DNA
ggaggaaatacaactcttgctg

SEQ ID NO: 70 - DNA, 18 bp, synthetic construct
  source 1..18: mol_type = other DNA
accacggtgtgcaaaaag

SEQ ID NO: 71 - DNA, 21 bp, synthetic construct
  source 1..21: mol_type = other DNA
ctgacaaggtagtaggtgagg

SEQ ID NO: 72 - DNA, 20 bp, synthetic construct
  source 1..20: mol_type = other DNA
gaatgaacggcttgcaaaac

SEQ ID NO: 73 - DNA, 22 bp, synthetic construct
  source 1..22: mol_type = other DNA
ccaaaacaatgccttcttcaac

SEQ ID NO: 74 - DNA, 21 bp, synthetic construct
  source 1..21: mol_type = other DNA
gagagattcaaccagttagcg

SEQ ID NO: 75 - DNA, 22 bp, synthetic construct
  source 1..22: mol_type = other DNA
actttccaagaatgtcaccatc

SEQ ID NO: 76 - DNA, 21 bp, synthetic construct
  source 1..21: mol_type = other DNA
tcatcgactacattctcgacc

SEQ ID NO: 77 - DNA, 21 bp, synthetic construct
  source 1..21: mol_type = other DNA
gtcgttggagatgacaagttc

SEQ ID NO: 78 - DNA, 21 bp, synthetic construct
  source 1..21: mol_type = other DNA
tgcagcaatcacatctacccc

SEQ ID NO: 79 - DNA, 20 bp, synthetic construct
  source 1..20: mol_type = other DNA
ttcccatcatgcctgcgttc

SEQ ID NO: 80 - DNA, 18 bp, synthetic construct
  source 1..18: mol_type = other DNA
aacaaagctgctgcaacc

SEQ ID NO: 81 - DNA, 19 bp, synthetic construct
  source 1..19: mol_type = other DNA
ccattagcgcatcacagtc

SEQ ID NO: 82 - DNA, 20 bp, synthetic construct
  source 1..20: mol_type = other DNA
aagaccaagggcaaccgaag

SEQ ID NO: 83 - DNA, 22 bp, synthetic construct
  source 1..22: mol_type = other DNA
ttgcagaagagccaggaggaac

SEQ ID NO: 84 - DNA, 20 bp, synthetic construct
  source 1..20: mol_type = other DNA
acactccaaagacggcagac

SEQ ID NO: 85 - DNA, 20 bp, synthetic construct
  source 1..20: mol_type = other DNA
aacacatccaagagggccac

SEQ ID NO: 86 - DNA, 20 bp, synthetic construct
  source 1..20: mol_type = other DNA
ctggattggacctcaatgac

SEQ ID NO: 87 - DNA, 20 bp, synthetic construct
  source 1..20: mol_type = other DNA
actccctgaagacatctcac

SEQ ID NO: 88 - DNA, 22 bp, synthetic construct
  source 1..22: mol_type = other DNA
gatcattgcacccaagggctat

SEQ ID NO: 89 - DNA, 22 bp, synthetic construct
  source 1..22: mol_type = other DNA
ttagcttagttggcgcacagca

SEQ ID NO: 90 - DNA, 19 bp, synthetic construct
  source 1..19: mol_type = other DNA
tcaaccgcgaccctaaaca

SEQ ID NO: 91 - DNA, 19 bp, synthetic construct
  source 1..19: mol_type = other DNA
aagctggccttccaaatgc

SEQ ID NO: 92 - DNA, 20 bp, synthetic construct
  source 1..20: mol_type = other DNA
gaattctctttgccaccctc

SEQ ID NO: 93 - DNA, 22 bp, synthetic construct
  source 1..22: mol_type = other DNA
tgtcacactcttccatattgtc

SEQ ID NO: 94 - DNA, 20 bp, synthetic construct
  source 1..20: mol_type = other DNA
acaagaagggctacctggac

SEQ ID NO: 95 - DNA, 21 bp, synthetic construct
  source 1..21: mol_type = other DNA
cctgaaaaggaggagtggatg

SEQ ID NO: 96 - DNA, 19 bp, synthetic construct
  source 1..19: mol_type = other DNA
ttcccgcagtaatgacacc

SEQ ID NO: 97 - DNA, 20 bp, synthetic construct
  source 1..20: mol_type = other DNA
cacagagctaaaagcccaac

SEQ ID NO: 98 - DNA, 19 bp, synthetic construct
  source 1..19: mol_type = other DNA
gatccgttacaagcgtcag

SEQ ID NO: 99 - DNA, 19 bp, synthetic construct
  source 1..19: mol_type = other DNA
actccagtcactcagttcc

SEQ ID NO: 100 - DNA, 22 bp, synthetic construct
  source 1..22: mol_type = other DNA
ggacatgagtcaaaactactgc

SEQ ID NO: 101 - DNA, 21 bp, synthetic construct
  source 1..21: mol_type = other DNA
ccgacgactgtgataagaaac

SEQ ID NO: 102 - DNA, 20 bp, synthetic construct
  source 1..20: mol_type = other DNA
agctggatgactacctgaac

SEQ ID NO: 103 - DNA, 19 bp, synthetic construct
  source 1..19: mol_type = other DNA
gcttgaatcctgatgccac

SEQ ID NO: 104 - DNA, 19 bp, synthetic construct
  source 1..19: mol_type = other DNA
tgacaacggatacccagac

SEQ ID NO: 105 - DNA, 19 bp, synthetic construct
  source 1..19: mol_type = other DNA
aggacacagctcttcacag

SEQ ID NO: 106 - DNA, 20 bp, synthetic construct
  source 1..20: mol_type = other DNA
tcagccccaatcccaatacc

SEQ ID NO: 107 - DNA, 20 bp, synthetic construct
  source 1..20: mol_type = other DNA
atgaccttcagcagcacctc

SEQ ID NO: 108 - DNA, 21 bp, synthetic construct
  source 1..21: mol_type = other DNA
atgcctacatctacgttgtcc

SEQ ID NO: 109 - DNA, 20 bp, synthetic construct
  source 1..20: mol_type = other DNA
actgttgtcccatcctcgtc

SEQ ID NO: 110 - DNA, 20 bp, synthetic construct
  source 1..20: mol_type = other DNA
cttaccacctactgaccacc

SEQ ID NO: 111 - DNA, 19 bp, synthetic construct
  source 1..19: mol_type = other DNA
accatcttccagagtgacc

SEQ ID NO: 112 - DNA, 20 bp, synthetic construct
  source 1..20: mol_type = other DNA
tcactctcaagcctggtaac

SEQ ID NO: 113 - DNA, 22 bp, synthetic construct
  source 1..22: mol_type = other DNA
ctctctcccactttttccattc

SEQ ID NO: 114 - DNA, 19 bp, synthetic construct
  source 1..19: mol_type = other DNA
agacaaagacccgcaaaag

SEQ ID NO: 115 - DNA, 19 bp, synthetic construct
  source 1..19: mol_type = other DNA
taaacaggacggaggtgac

SEQ ID NO: 116 - DNA, 20 bp, synthetic construct
  source 1..20: mol_type = other DNA
gacatcagcaagtatggcag

SEQ ID NO: 117 - DNA, 20 bp, synthetic construct
  source 1..20: mol_type = other DNA
tcctttctcagcttctccac

SEQ ID NO: 118 - DNA, 19 bp, synthetic construct
  source 1..19: mol_type = other DNA
gcacacctcgtaacttctc

SEQ ID NO: 119 - DNA, 19 bp, synthetic construct
  source 1..19: mol_type = other DNA
caccacagcgtttaaccag

SEQ ID NO: 120 - DNA, 19 bp, synthetic construct
  source 1..19: mol_type = other DNA
cgaagtcaagagccacaag

SEQ ID NO: 121 - DNA, 20 bp, synthetic construct
  source 1..20: mol_type = other DNA
ttccgataccccagatgaag

SEQ ID NO: 122 - DNA, 20 bp, synthetic construct
  source 1..20: mol_type = other DNA
actccatctcagaccccttc

SEQ ID NO: 123 - DNA, 20 bp, synthetic construct
  source 1..20: mol_type = other DNA
gccttcgtggttgttttctc

SEQ ID NO: 124 - DNA, 20 bp, synthetic construct
  source 1..20: mol_type = other DNA
ccaacccactacagacagac

SEQ ID NO: 125 - DNA, 20 bp, synthetic construct
  source 1..20: mol_type = other DNA
tatcacagacatcccccacc

SEQ ID NO: 126 - DNA, 21 bp, synthetic construct
  source 1..21: mol_type = other DNA
tctcacctatcgcaacctatc

SEQ ID NO: 127 - DNA, 19 bp, synthetic construct
  source 1..19: mol_type = other DNA
ccaaaggcatgacacagac

SEQ ID NO: 128 - DNA, 20 bp, synthetic construct
  source 1..20: mol_type = other DNA
aggctttcccaagcaaatag

SEQ ID NO: 129 - DNA, 22 bp, synthetic construct
  source 1..22: mol_type = other DNA
cttcaaagtgtgtgtcactttc

SEQ ID NO: 130 - DNA, 20 bp, synthetic construct
  source 1..20: mol_type = other DNA
attactcctcagaaccccag

SEQ ID NO: 131 - DNA, 21 bp, synthetic construct
  source 1..21: mol_type = other DNA
ccccatctacagaaccttcag

SEQ ID NO: 132 - DNA, 22 bp, synthetic construct
  source 1..22: mol_type = other DNA
gaatgtcactctaatccctgtc

SEQ ID NO: 133 - DNA, 20 bp, synthetic construct
  source 1..20: mol_type = other DNA
aatttgtgccagccatagtc

SEQ ID NO: 134 - DNA, 21 bp, synthetic construct
  source 1..21: mol_type = other DNA
ttccactttgctaatgactcc

SEQ ID NO: 135 - DNA, 19 bp, synthetic construct
  source 1..19: mol_type = other DNA
gcattgacagatgtcagcc

SEQ ID NO: 136 - DNA, 21 bp, synthetic construct
  source 1..21: mol_type = other DNA
gggaatgggacaaaaagacag

SEQ ID NO: 137 - DNA, 19 bp, synthetic construct
  source 1..19: mol_type = other DNA
aggcatagagagacagcac

SEQ ID NO: 138 - DNA, 22 bp, synthetic construct
  source 1..22: mol_type = other DNA
ctggcctagctcgatattatcc

SEQ ID NO: 139 - DNA, 20 bp, synthetic construct
  source 1..20: mol_type = other DNA
acacttccactcacatccac

SEQ ID NO: 140 - DNA, 20 bp, synthetic construct
  source 1..20: mol_type = other DNA
gtatgtgaacactgtgaccc

SEQ ID NO: 141 - DNA, 21 bp, synthetic construct
  source 1..21: mol_type = other DNA
gcaaaagaagaagacaaagcc

SEQ ID NO: 142 - DNA, 20 bp, synthetic construct
  source 1..20: mol_type = other DNA
agcagttctacaatgccatc

SEQ ID NO: 143 - DNA, 19 bp, synthetic construct
  source 1..19: mol_type = other DNA
taaccttcgcccaacagtc

SEQ ID NO: 144 - DNA, 19 bp, synthetic construct
  source 1..19: mol_type = other DNA
ttccaactgccaaagccac

SEQ ID NO: 145 - DNA, 20 bp, synthetic construct
  source 1..20: mol_type = other DNA
atgacctctgccttctgtcc

SEQ ID NO: 146 - DNA, 20 bp, synthetic construct
  source 1..20: mol_type = other DNA
catgttccagactgttgacc

SEQ ID NO: 147 - DNA, 21 bp, synthetic construct
  source 1..21: mol_type = other DNA
cctgcgctttcttcataaacc

SEQ ID NO: 148 - DNA, 20 bp, synthetic construct
  source 1..20: mol_type = other DNA
aacctgaccctaatggcgac

SEQ ID NO: 149 - DNA, 20 bp, synthetic construct
  source 1..20: mol_type = other DNA
atatgtggcaggaggcgtag

SEQ ID NO: 150 - DNA, 22 bp, synthetic construct
  source 1..22: mol_type = other DNA
gcaaactgctggacatcgcttg

SEQ ID NO: 151 - DNA, 23 bp, synthetic construct
  source 1..23: mol_type = other DNA
gccatagtggatgtcaaacgagg

SEQ ID NO: 152 - DNA, 21 bp, synthetic construct
  source 1..21: mol_type = other DNA
cgactatgatgagacctggac

SEQ ID NO: 153 - DNA, 19 bp, synthetic construct
  source 1..19: mol_type = other DNA
gtagcgaaaggtgtagaag

SEQ ID NO: 154 - DNA, 22 bp, synthetic construct
  source 1..22: mol_type = other DNA
ggtggcttcagcaatgtgttcc

SEQ ID NO: 155 - DNA, 22 bp, synthetic construct
  source 1..22: mol_type = other DNA
gaagtaactggatggtggcagg

SEQ ID NO: 156 - DNA, 20 bp, synthetic construct
  source 1..20: mol_type = other DNA
agcaactgagacagcaacca

SEQ ID NO: 157 - DNA, 21 bp, synthetic construct
  source 1..21: mol_type = other DNA
agacgtaccagccatttgtgt

SEQ ID NO: 158 - DNA, 22 bp, synthetic construct
  source 1..22: mol_type = other DNA
ttcagtaaccaagtcttccaac

SEQ ID NO: 159 - DNA, 22 bp, synthetic construct
  source 1..22: mol_type = other DNA
cagcattctctttcacaacttc

SEQ ID NO: 160 - DNA, 20 bp, synthetic construct
  source 1..20: mol_type = other DNA
ctcgtatatcgcgctcatca

SEQ ID NO: 161 - DNA, 20 bp, synthetic construct
  source 1..20: mol_type = other DNA
tcttgacgaagcagtcgttg

SEQ ID NO: 162 - DNA, 19 bp, synthetic construct
  source 1..19: mol_type = other DNA
cccaaaaatcctcgctcac

SEQ ID NO: 163 - DNA, 20 bp, synthetic construct
  source 1..20: mol_type = other DNA
agtccgtttcgttcaagttc

SEQ ID NO: 164 - DNA, 22 bp, synthetic construct
  source 1..22: mol_type = other DNA
caatctctctccaccttttgtc

SEQ ID NO: 165 - DNA, 19 bp, synthetic construct
  source 1..19: mol_type = other DNA
tgcccatatccatgtcacc

SEQ ID NO: 166 - DNA, 19 bp, synthetic construct
  source 1..19: mol_type = other DNA
tccaccaccaaacatgcag

SEQ ID NO: 167 - DNA, 19 bp, synthetic construct
  source 1..19: mol_type = other DNA
aagggacacaacgacacac

SEQ ID NO: 168 - DNA, 19 bp, synthetic construct
  source 1..19: mol_type = other DNA
ccctcctttgctgaaatcc

SEQ ID NO: 169 - DNA, 20 bp, synthetic construct
  source 1..20: mol_type = other DNA
ctccttttccacttcgtctg

SEQ ID NO: 170 - DNA, 20 bp, synthetic construct
  source 1..20: mol_type = other DNA
cagatgcgagagatcctgag

SEQ ID NO: 171 - DNA, 22 bp, synthetic construct
  source 1..22: mol_type = other DNA
gccaaagaagacgatgaagtag

SEQ ID NO: 172 - DNA, 20 bp, synthetic construct
  source 1..20: mol_type = other DNA
cgtgatagaagagctgttgg

SEQ ID NO: 173 - DNA, 20 bp, synthetic construct
  source 1..20: mol_type = other DNA
cttgactactgagcatcacc

SEQ ID NO: 174 - DNA, 21 bp, synthetic construct
  source 1..21: mol_type = other DNA
gtcttcaactattacgacccc

SEQ ID NO: 175 - DNA, 21 bp, synthetic construct
  source 1..21: mol_type = other DNA
acttttcactgtgttcttccc

SEQ ID NO: 176 - DNA, 20 bp, synthetic construct
  source 1..20: mol_type = other DNA
accagattcttggcagaagg

SEQ ID NO: 177 - DNA, 20 bp, synthetic construct
  source 1..20: mol_type = other DNA
tgcagaccagcctggataaa

SEQ ID NO: 178 - DNA, 21 bp, synthetic construct
  source 1..21: mol_type = other DNA
cctggtgctccatgaggagac

SEQ ID NO: 179 - DNA, 22 bp, synthetic construct
  source 1..22: mol_type = other DNA
cagactctgaccttttgccagg

SEQ ID NO: 180 - DNA, 20 bp, synthetic construct
  source 1..20: mol_type = other DNA
tcaatgccaacaaggaagag

SEQ ID NO: 181 - DNA, 20 bp, synthetic construct
  source 1..20: mol_type = other DNA
acacgacagaaagaagcaag

SEQ ID NO: 182 - DNA, 21 bp, synthetic construct
  source 1..21: mol_type = other DNA
gatgatgaatacgaggtggac

SEQ ID NO: 183 - DNA, 19 bp, synthetic construct
  source 1..19: mol_type = other DNA
tgatggtgatgaggatggg

SEQ ID NO: 184 - DNA, 20 bp, synthetic construct
  source 1..20: mol_type = other DNA
aagcagcagtaccgagtacc

SEQ ID NO: 185 - DNA, 20 bp, synthetic construct
  source 1..20: mol_type = other DNA
tctcacagacatccacagcc

SEQ ID NO: 186 - DNA, 21 bp, synthetic construct
  source 1..21: mol_type = other DNA
ccgtgaatgctaaagttctcc

SEQ ID NO: 187 - DNA, 22 bp, synthetic construct
  source 1..22: mol_type = other DNA
gacacacacactaagatgacag

SEQ ID NO: 188 - DNA, 20 bp, synthetic construct
  source 1..20: mol_type = other DNA
ctgaacggaccgctatccag

SEQ ID NO: 189 - DNA, 21 bp, synthetic construct
  source 1..21: mol_type = other DNA
aggccccacatggagagataa